(19) United States
(12) Patent Application Publication
(10) Pub. No.: US 2026/0315822 A1
(43) Pub. Date: Oct. 15, 2026

(54) Unibody Apparatus for Phototherapy

(71) Applicant: INCANDO THERAPEUTICS PTE. LTD., Singapore (SG)

(72) Inventors: Percy Luu, Singapore (SG); Cheng-Wei Pei, Belmont, CA (US); Tsang Chung Charles Hu, Stanley (HK); Reghu Kunnath, Whittier, CA (US); James Marshall Robinson, Hyattsville, MD (US)

(21) Appl. No.: 19/114,359

(22) PCT Filed: Sep. 19, 2023
(86) PCT No.: PCT/US2023/033104

§ 371 Date: Mar. 21, 2025

Related U.S. Application Data
(60) Provisional application No. 63/410,676, filed on Sep. 28, 2022.

(51) Int. Cl. A61N 5/06; A61N 5/00

(52) U.S. Cl. CPC A61N 5/062; A61N 2005/005; A61N 2005/0651; A61N 2005/0658; A61N 2005/0664

(57) ABSTRACT

A device and method for phototherapy includes a light source and an encapsulant. The light source is encapsulated by the encapsulant. Light is emitted from the light source to treat target tissue and the encapsulant isolates the light source or other components in the encapsulant from adjacent tissue. The method for phototherapy comprises: illuminating a target treatment tissue with light emitted from a device comprising a light source, wherein the light source is encapsulated in an encapsulant; and maintaining isolation between the light source and the target treatment tissue with the encapsulant.

### CLAIM OF PRIORITY

This patent application claims the benefit of priority to U.S. Provisional Patent Application Ser. No. 63/410,676, filed on Sep. 28, 2022 (Attorney Docket No. 5405.002PRV), the entire contents of which are incorporated herein by reference.

### BACKGROUND

Implanted electronics in medical devices may require protection from the body's fluids, and the body requires protection from any harmful or potentially harmful materials used in those implanted devices. Therefore, components of implanted medical devices use materials that are proven to be safe in the body. For those components that are either not proven safe or are susceptible to the body's fluids, those components can still be used in certain situations such as when they are functionally isolated to prevent contact with the body and any adjacent tissue.

Examples of implantable electronics are disclosed herein that may address some of these challenges. While the examples will emphasize the ability to deliver light to tissue in the body, this is not intended to be limiting and one of skill in the art will appreciate that other applications of the examples disclosed herein are possible.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates an example of encapsulated heat generating electronics.

FIG. 2A shows a block diagram of a light delivery device.

FIG. 2B shows a schematic of the light delivery device in FIG. 2A.

FIG. 2C shows an example of the light delivery device in FIGS. 2A-2B.

FIG. 3 shows another example of an encapsulated light source.

FIG. 4 shows an example of multiple light sources encapsulated.

FIG. 5 shows an example of a light delivery device with refraction structures.

FIG. 6 illustrates an example of a light delivery device that emits light in multiple directions.

FIG. 7 illustrates an example of a light delivery device that emits light from a side surface.

FIG. 8 illustrates another example of a light delivery device that is encapsulated.

FIG. 9 illustrates an example of a device that includes an encapsulated light.

FIG. 10 shows an example of a device with encapsulated sensors.

FIG. 11 shows an example of a device with an encapsulated optical sensor.

FIG. 12 shows an example of an encapsulated device that permits capacitive sensing.

FIG. 13 shows an example of an encapsulated device with a coil.

FIG. 14 shows an example of an encapsulated device with capacitive properties.

FIG. 15 shows an example of an encapsulated light emitter device.

FIG. 16 shows an example of implantation of a light emitter device in the brain.

FIGS. 17A-17B show other examples of implantation of one or more light emitter devices in the brain.

FIG. 18 illustrates transnasal delivery of a light emitter device to the brain.

FIGS. 19A-19E show examples of light intensity waveforms that may be used to treat target tissue.

FIG. 20 shows an example of an implantable illumination device.

FIG. 21 shows an example of implantation of an illumination device through a burr hole in the skull.

FIG. 22 shows an example of an encapsulated illumination device.

FIG. 23 shows another example of an encapsulated illumination device.

FIGS. 24A-24B show a perspective and cross-section of the device in FIG. 23.

FIGS. 25A-25C show examples of illumination devices with distal shapes to control light distribution.

FIGS. 26A-26C show an example of a burr cap that may be used with an illumination device.

FIG. 27 shows an example of a burr cap.

FIGS. 28A-28B show an example of a securement plate.

FIGS. 29A-29B show examples of devices for cable or tether tensioning in an illuminated device.

FIG. 30 shows an example of bilateral devices implanted in the brain.

FIG. 31 shows an example of an illumination device that may illuminate multiple areas of target tissue.

FIG. 32 shows an example of an illumination device with structures for guiding light.

FIG. 33 shows an illumination device with a hybrid tether.

FIG. 34 shows an illumination device with a lumen for drug delivery.

FIG. 35 shows a block diagram of electronics which may be used in an illumination device.

FIGS. 36A-36C illustrate surgical instruments which may be used to deliver an illumination device to a target treatment area.

FIG. 37 shows an example of an illumination device with an expandable member.

### DETAILED DESCRIPTION

Light delivery as a therapeutic is an integral part of human existence. Light from the sun helps regulate our circadian rhythm and produce crucial Vitamin D in our skin throughout the day. Light is used in the form of therapy to treat conditions of the eyes and skin, or to reduce bilirubin levels to treat newborn jaundice. These optical devices for receiving and transmitting light, including photodiodes, thermopiles, lasers, light-emitting diodes (LEDs), photomultiplier tubes (PMTs), avalanche photodiodes (APDs), and the like often require additional optical elements to aid their primary function. These optical elements can, among other functionality, focus, redirect, amplify, reduce, diffuse, or filter the incoming or outgoing light.

Examples of elements that may be used to control light include lenses, optical filters (including neutral density filters), beam splitters, prisms, diffusers, diffraction gratings, apertures, and mirrors. Some of these elements require precise alignment and/or orientation to ensure performance.

Mechanical encapsulation of optoelectronic devices to insulate them from a living body constrains the ability of a product designer to control light, because the encapsulation itself will be in the optical path, and because the encapsulation materials may be unsuited for use in optical applications. Encapsulation materials may include titanium, ceramic (including glass, sapphire), alumina-based ceramics (Al₂O₃), zirconia-based ceramics (YTZP, ATZ, ZTA) and stainless steel.

This disclosure describes examples of optically active, monolithic or unibody encapsulation structures, light delivery, and integration aspects with other devices of such implants. The implant can also be designed such that the monolithic encapsulation materials are formulated, designed, and/or shaped to act as optical filters, lenses, refractors, diffusers, or other traditional optical elements. The elimination of additional optical elements reduces space, cost, and weight from the final device design. Furthermore, thermo-compression bonding and thermo-forming can be applied to create hermetically sealed devices with a surface-conforming shape. Multi-functional elements, such as tissue interfaces and microelectronic packaging, can be integrated on the same monolithic encapsulation substrate to make it an attractive material for implantable devices. Micro-fabrication technologies such as metal deposition, photolithography, wet etching, dry (plasma) etching, and laser machining can be utilized on monolithic encapsulation substrates. The tissue conformable structure is another advantage of this encapsulation approach. Desired surface shapes can be thermally formed using molds. The RF (radiofrequency) transparence of such monolithic encapsulation substrates allows coils to be integrated inside the packaging, minimizing the overall size of a device. The energy efficiency is increased because the coil is well encapsulated, allowing highly RF-efficient metals such as copper to be used.

A hermetic seal may be needed to ensure that fluids and gases can't penetrate the system package encapsulation over the necessary time frame, which could be the entire life span of a patient who receives an implant.

The main challenge of using light to treat internal diseases is that light does not travel very far into the body. Light is absorbed by the skin and tissue, which limits the penetration depth of visible and near-infrared (NIR) wavelengths to 3 to 5 millimeters. In some instances, applying phototherapy to tumors or stimulating neurons to treat movement disorders may require light at 10-25 cm depths, less depth, or even greater depths, and an implantable light source adjacent the target treatment area is the only practical way for light to reach such depths in the human body.

The implant can also be designed to deliver a single treatment and/or integrated with other forms of stimulation and/or therapeutic agents other than light, and in doing so deliver innovative combination therapies. The implant can also be modified to include sensing properties, such as to modulate treatment dosage in response to the patient's physiological state. An implant can be enabled to intervene and/or interact with one or more functions of the body by sensing and/or stimulating aspects of body physiology, which requires bi-directional transfer of energy and signals between the implanted electronics and the surrounding body tissue. Light delivery provided via a monolithic encapsulation device may be used to treat numerous conditions throughout the body. Non-limiting examples of conditions which may be treated using the examples of devices disclosed herein include but are not limited to treating brain tumors such as glioblastomas, Parkinson's Disease, or any other damaged or diseased tissue including other neurodegenerative conditions in the body.

The following, non-limiting examples, detail certain aspects of the present subject matter to solve at least some of the challenges and provide the benefits discussed herein, among others.

This disclosure describes certain aspects of using specific material properties of an encapsulant, and adding materials to the encapsulation of medical devices, to alter and enhance certain properties at specific areas of the implant, while remaining biocompatible for the purposes of temporary or permanent contact with a living body. This approach applies to clinical tools, disposable single-use medical devices, devices that are reprocessed for multiple use, and permanently implanted devices. The same considerations apply to both animal and human medical devices.

There are a variety of encapsulation materials that can be relevant for implementing the devices described, including thin-film inorganic coatings of aluminum oxide, Hafnium(IV) oxide, Silicon dioxide, silicon carbide, and diamond, as well as organic polymers of polyimide, parylene, liquid crystal polymer, silicone elastomer, SU-8 (SU-8 is a highly crosslinked amorphous polymer built of monomers with eight epoxy binding sites wherein one monomer is based on four bisphenol-A units connected to each other at the phenyl rings), and cyclic olefin copolymer. Crystalline ceramics including glass and sapphire also have good optical properties, and some examples may include these materials as well. Suitable materials for a long-term implantable device will have the following properties: flexible, mechanically stable, high Young's moduli, and biocompatible materials that have a much lower moisture absorption rate (less than 0.04%, comparable to PTFE and borosilicate glass) compared to polyimide and parylene-C. These materials have suitable barrier properties against various chemicals and are capable of being thermally bonded to each other without adhesives. These monolithic encapsulation materials demonstrate high reliability under harsh environmental conditions. For example, liquid-crystal materials have many advantages in mechanical and chemical properties such as extremely low water absorbability, chemical stability, simultaneous flexibility and rigidity, low dielectric constant, and compatibility with micro-fabrication process. Thermotropic liquid-crystal materials consist of anisotropic molecules that self-assemble into phases with orientational order, but often no positional order, in their simplest form. These phases exist between the conventional crystalline phase and the isotropic liquid phase. The molecules show a high degree of shape anisotropy (for example, rod-like or disk-like), which manifests itself in many ways, such as dielectric and optical anisotropies. The material may pass through one or many different liquid-crystal phases, characterized by order and symmetry, before transforming into a truly isotropic fluid (the liquid phase).

In all the drawings, the light sources shown may comprise light-emitting diodes (LED), laser diodes, organic LED (OLED), or other optoelectronic devices. They may also be light-receiving devices such as photodiodes, photomultiplier tubes, avalanche photodiodes, thermopiles, or other components that convert incident light into an electronically quantifiable form. Therefore, the light source may be any source of light known in the art.

Also, for purposes of brevity, every drawing does not show electrical routing, for example using gold or copper traces within the encapsulant itself or an embedded substrate printed circuit board (PCB) that serves the purpose of electrically connecting the LED to the rest of an electronic circuit.

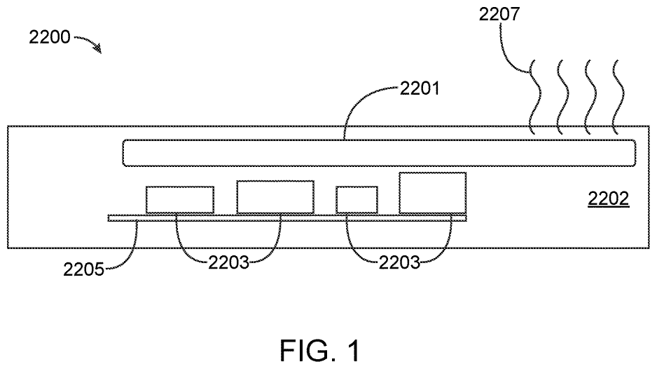

FIG. 1 shows an example of a device 2200 with electronic or other components 2203 which generate heat that are embedded in an encapsulant. The encapsulant in this example or any example disclosed herein may be any of the encapsulants disclosed in this specification or otherwise known in the art. Here, the components may be any active electronic components which generate heat. Components such as LEDs, which are not 100% efficient, may dissipate more heat than certain other components. It is important for active medical implants to provide adequately low threshold thermal resistance and spread the generated heat such that the rise in temperature is limited. Certain encapsulants, which can penetrate, and form contact surfaces with components may sink and disperse heat to the surface of an implant in contact with body tissue to help dissipate the heat and control the rise in temperature. Such encapsulants may also provide thermal insulation to prevent point-heating of the tissue.

Here, the device 2200 may include electronic components 2203 which may be any electronic components (e.g., LED's, processors, resistors, capacitors, memory chips, etc.) including components that generate heat. There may be one or more electronic components and they may optionally be mounted on a substrate 2205 such as a printed circuit board (PCB). The device optionally includes an embedded material 2201 with low thermal resistance which absorbs heat and helps disperse the heat over a larger surface area and thus avoid hot spots on the biological tissue when the device is implanted in tissue. The entire device may then be encapsulated in an encapsulant material 2202. The heat 2207 can then dissipate from the encapsulated device. Thus, the encapsulant in this example or any encapsulated example disclosed herein can act as a heat dispersion aid and/or an insulation material.

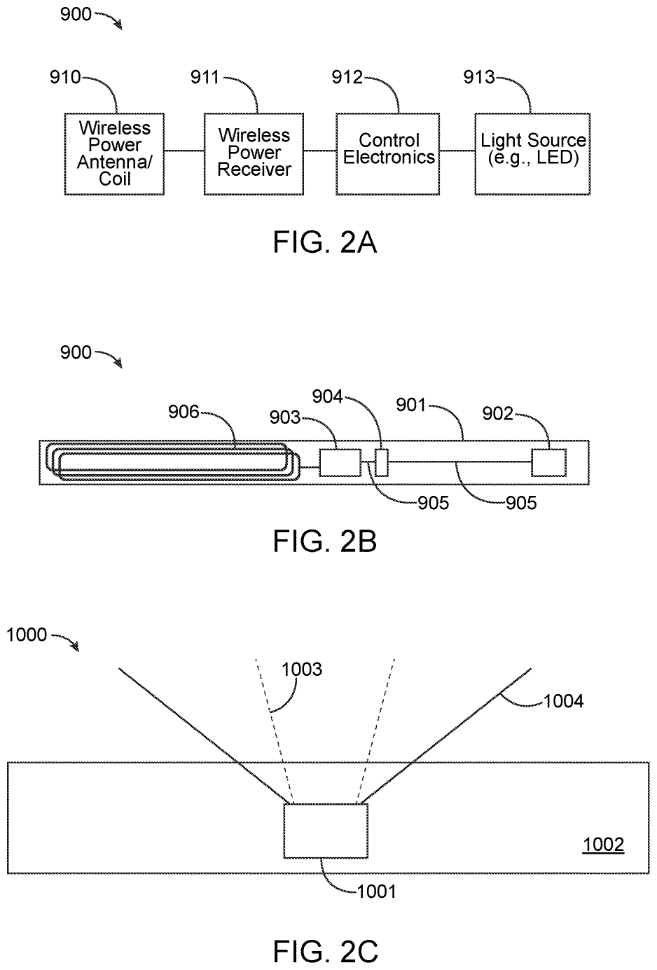

FIG. 2A shows a block diagram of an example of a light delivery device 900 which may be implanted in tissue in order to provide illumination in a patient. The system may include a wireless power antenna or coil 910 for receiving energy from another source such as an external source. The energy is then received by a wireless power receiver 911. Control electronics 912 then control operation of the device to turn on the light source and deliver light or turn off the light source 913.

FIG. 2B illustrates an example of the device 900 described above in FIG. 2A. Here, a wireless power antenna 906 receives power and then transfers the power to a wireless power receiver 903 which may comprise one or more integrated circuits and other electronic components. Additional control electronics 904 may be required for power management, communication, and control of the light source. The light source itself 902 emits light at wavelengths appropriate for the type of phototherapy desired. The circuitry is connected by wiring 905 which may be in a printed circuit board. The entire system can be encapsulated by an encapsulation material 901, which could be polyimide, liquid crystal polymer, or any other material disclosed herein.

FIG. 2C illustrates an example of the light source 902 in FIGS. 2A-2B. The light source may be an LED 1001 which is encapsulated in a monolithic encapsulant 1002. The encapsulant material may have the properties of being an optical diffuser, such that the inherent light output pattern 1003 of the LED prior to encapsulation is broadened (represented by 1004). The optical absorption characteristic of the encapsulant is not shown but may be minimized while maximizing the scattering/refractive properties. The encapsulant material and shape or geometry of the encapsulant surrounding the light source may be selected in order to provide desired optical properties.

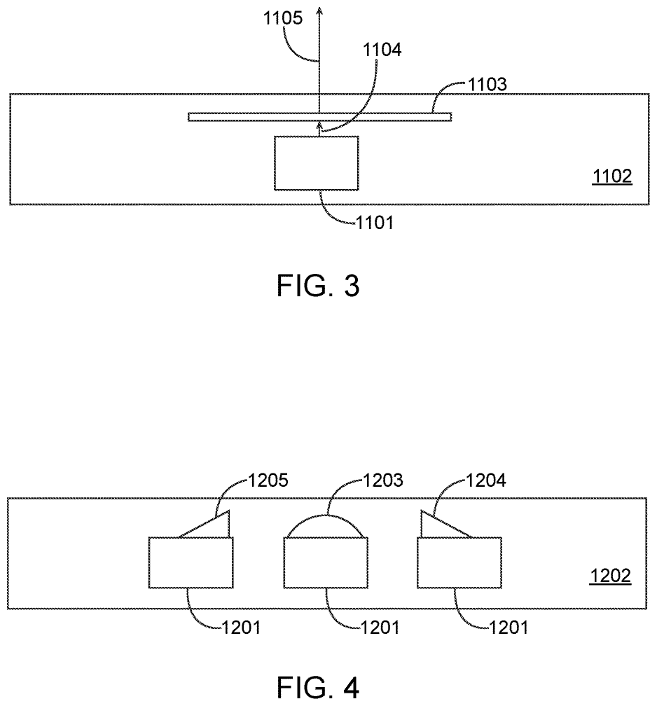

FIG. 3 shows another example of an encapsulated light source. Here, the light source, e.g., LED 1101 is shown in an encapsulant 1102. The light emitted from the LED is represented by 1104 and may pass into an optional second embedded optical element (1103) which may have additional desired optical properties to modify light output. The resulting light output is altered, represented by 1105.

The optical element 1103 can modify the light by changing the spectrum, as an optical filter, for example an absorptive polymer or thin film-based material that absorbs some wavelengths and passes others. The filter could also be a multi-layer dielectric filter, that uses constructive/destructive interference to block some wavelengths and pass others. The multi-layer dielectric filter could also be dynamic, e.g., a microelectromechanical (MEMS) tunable cavity filter, or MEMS shutter-based filter.

The optical element 1103 could also convert the light, for example, to emits light at different wavelength(s) than the incoming light. The layer could comprise phosphors such as those used in white LED products.

FIG. 4 shows multiple light sources 1201 that are encapsulated in a material 1202. Encapsulated together with the light source are optical elements that refract the light, such as lenses or angled refractors (1203, 1204, 1205). These shaped structures could also be formed from air, in which case the encapsulant must be applied in layers and the shapes (1203, 1204, 1205) molded into the structures. Thus, the optical elements may be used to shape the light by focusing or diffusing it, or direct the light into a desired direction.

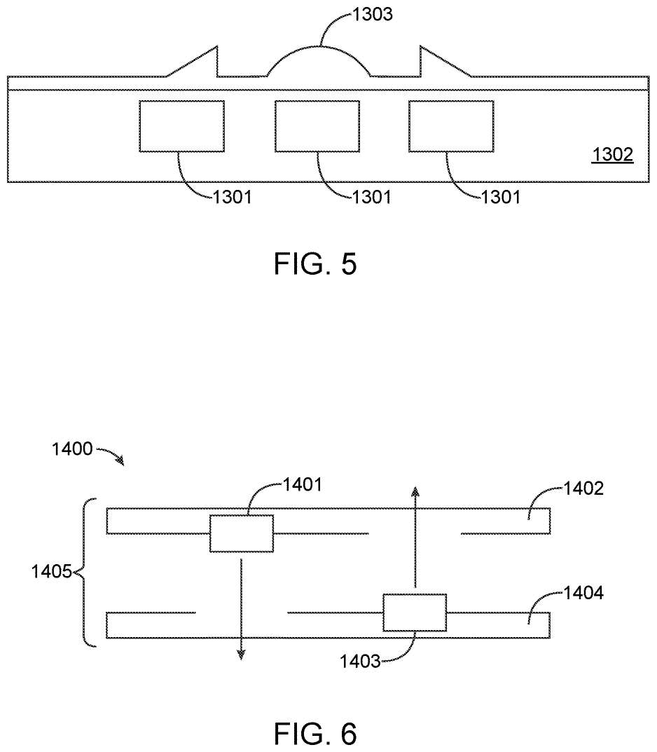

FIG. 5 shows that the refraction structures can also be molded into a second layer of material on the outside of the encapsulant, as shown by surface features 1303 which may be lenses or angled refractors or other structures. The second layer may be formed with material such as silicone, polyimide, or any other material that is biocompatible and has the desired optical properties. The encapsulant 1302 serves to isolate the light sources 1301 from the body after implantation, so the material 1303 can be optimized for optical qualities such as transparency. The material 1303 can also be combined with or impregnated with other materials such as optical diffuser particles. The refractive index of the material 1303 can also be optimized to act as an optical intermediary between the encapsulant 1302, and living tissue or fluids.

FIG. 6 shows a device 1400 manufactured with LEDs pointed in multiple directions. The light sources 1401 and 1403 are shown, each with a partial encapsulation 1402 and 1404 respectively. Each may be electrically connected to its own substrate (not shown). The arrows denote the direction of emitted light, thus one light source 1401 emits light downward, while the other light source 1403 emits light upward away from the first light source in the opposite direction. When the two substrates are pressed together, the combined structure 1405 will have light emitting in multiple directions. This concept splits the device into two similar pieces, and then recombines them during the final assembly steps.

FIG. 7 shows an example of an encapsulated illumination device where the LED 1501 may emit light from the side of the illumination source (axially along the longitudinal axis of the device in this example) or may even be mounted in a rotated orientation within the encapsulant 1502. The emitted light is in the direction of the lightguide structure 1503. This structure 1503, which may be made of a different type of polymer or crystalline material such as LCP (liquid crystal polymer), sapphire, or glass, may be rigid or semi-flexible and guides the light out of the structure. One benefit is that the structure can be physically smaller in the location of the lightguide element.

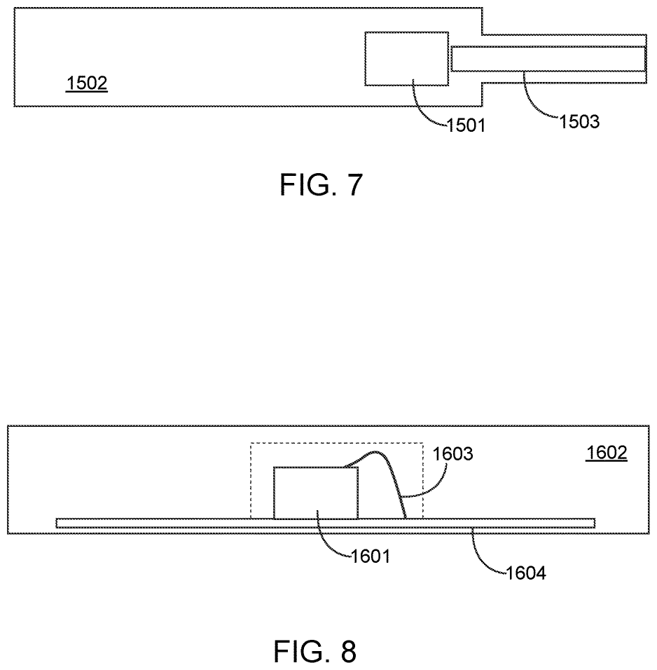

FIG. 8 shows an example of an encapsulated illumination device where the light source 1601 is shown in an encapsulant, 1602, which contains a cavity (indicated by the dotted line) in which the light source is disposed. A wire bond, 1603, electrically connects the light source 1601 to a substrate 1604 such as a printed circuit board. The purpose of the substrate 1604 is for electrical connections between the light source and the rest of the circuitry, and mechanical stability.

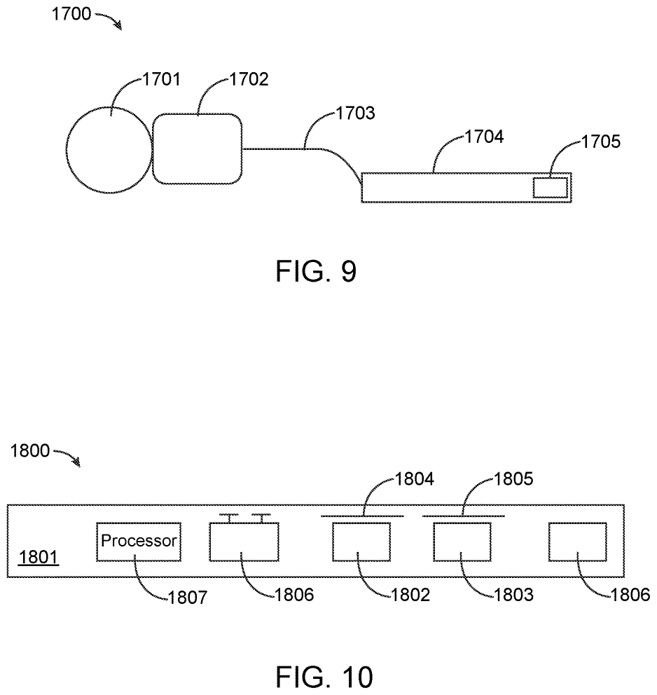

FIG. 9 shows an example of a device 1700 where the encapsulated light source can also be only a portion of a whole device. The encapsulation structure 1704 embeds a light source component 1705, but 1704 is also electrically connected to a wire 1703 which carries power and data signals to and from a central electronics module 1702. Electronics module 1702 may be integrated with a wireless power antenna/coil 1701 for receiving power and data from an external device (not shown), or it may be separate as shown. In this described architecture, encapsulant 1704 serves mainly to encapsulate the light source in a way that is biocompatible, for implanted devices.

FIG. 10 shows an example of a device 1800 with one or more sensors integrated directly into the encapsulation allowing the device to become more adaptive. The encapsulation 1801 encases multiple optional components in this example, including a processing unit 1807 and multiple sensors. One or more sensors 1806 may be an electrical sensor, for example using capacitance or radio-frequency sensing principles to detect something about the outside environment. Components 1802 and 1803 represent a light emitter and a sensor such as a light receiver, for example an LED and photodiode respectively. Additional optional optical elements 1804 and 1805 represent optical filters to reduce interference from other sources of light, or to block the “carrier” light wavelength in the case of fluorescence detection. By combining information from multiple sensors, the processor can utilize artificial intelligence and/or machine learning techniques to better control the amount and type of phototherapy delivered through light source 1806. Actuated control parameters for light source 1806 can include intensity, modulation (e.g., pulse width, modulation shape and depth, duty cycle, frequency), or repetition rate.

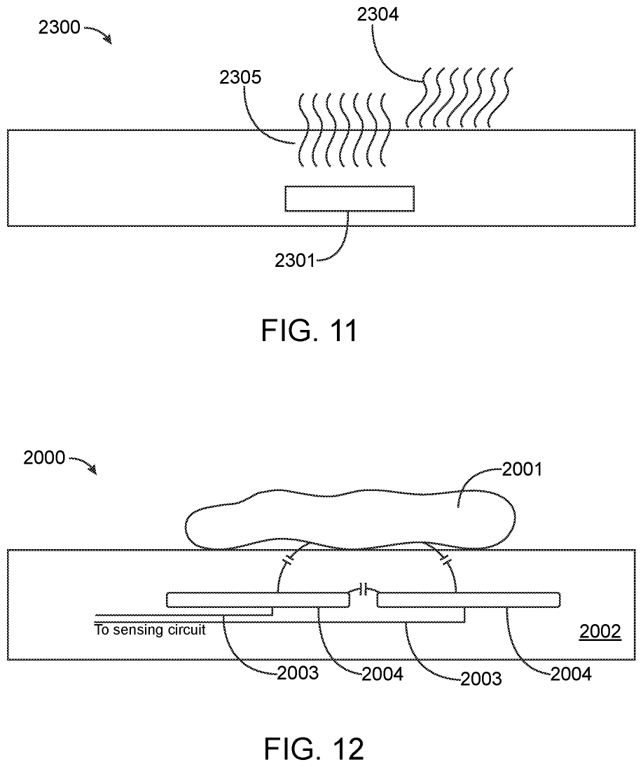

FIG. 11 shows an example of a device 2300 with an encapsulated optical sensor 2301 that may be used for optical sensing. The embedded optical sensor 2301 can be used to sense light of different wavelengths or from different physical directions to distinguish the sensed signal from any light transmitted out of the same device. For example, light-blocking or light-guiding structures or light filters can be used to preferentially allow light only from certain angles to strike the sensing surface of the sensor. Optical filters can be used to preferentially pass some wavelengths or polarizations of light, and block others reaching the sensor. The encapsulant may have optical properties such as selective wavelength filtering 2305 and directional selectivity 2304 to further ensure that the sensor receives the desired optical signal.

An encapsulated device may have capacitive properties such as capacitive sensing and stimulation through the encapsulant layer. A capacitor is formed when a dielectric is situated between two conductors. For an active implant with an encapsulant material, an active metallic surface inside the implant, that is connected to the implant circuits, forms a capacitor with the conductive body tissue with an insulator layer such as a layer of LCP as the dielectric. The apparent capacitance is directly related to the surface areas of the conductor and the area of the metallic conductor inside the implant, along with the tissue properties.

Such a capacitor may be used to sense signals from the tissue and stimulate electrical signals into the tissue. This approach may also be used to sense the presence of specific types of tissues on the surface of the implant that could affect the functioning of the implant, such as scar tissue or fibrosis.

FIG. 12 shows an example of an embedded capacitor in encapsulant 2002 and it has a buried capacitor formed from two parallel plates 2004. The fringe capacitance formed between the two plates is enhanced and varied by dielectric properties of objects 2001 on the surface of the implant, such as adjacent tissue. The buried circuit traces 2003 are connected to internal circuits to sense this capacitance to provide the ability to detect objects outside the implant.

Encapsulation may also be used to enhance electromagnetic properties of an embedded wireless coil or antenna. Encapsulant materials may be non-magnetic and allow embedded coils and antennas to be used for wireless power and data transfer. The capability to embed other materials in the encapsulant may also be utilized to enhance RF and electro-magnetic properties for more efficient communication and power transfer.

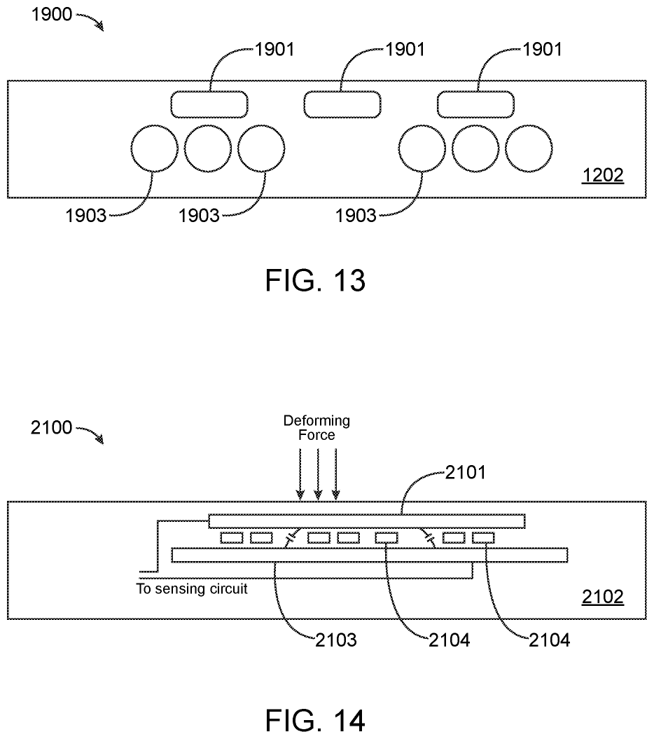

FIG. 13 shows an example device 1900 where the use of embedded material 1901 disposed in encapsulant 1202 is in the vicinity of a coil formed by conductors 1903. This coil may act as an inductive element or a tuned RF antenna. Based on the electromagnetic properties of the embedded materials 1901, the RF properties of the coil can be modulated to enhance the performance of the device such as improving the RF and electromagnetic properties of the device. Some examples of embedded material 1901 may include metallic materials or otherwise having magnetic properties, that allow the materials to shape a nearby magnetic field. This may include MOF materials. MOFs are crystalline materials composed of metal ions and organic linkers. MOFs have a high surface area and can be functionalized with a variety of molecules, including dyes and fluorescent molecules. When MOFs are added to hydrogels, they can be used to create hydrogels with tunable optical properties, such as color, reflectance, and transparency.

Embedding a device in an encapsulant may also create capacitive properties that can be used for other purposes, such as by forming a strain gauge because of the dielectric properties of the encapsulant. This enables the embedding of capacitors with buried conductors that may form capacitance. As the encapsulant material is flexible, deforming forces may readily change the electrical properties of the formed capacitance, enabling circuit sensing thus forming a capacitive strain gauge. This may be used to remotely assess the condition of the implant and forces exerted by the surrounding tissue.

FIG. 14 shows an example of an embedded device 2100 having capacitive properties. Here, a buried capacitor comprises two plates 2101, 2103. The capacitance formed is a function of the gap between the plates, area of the plates and the dielectric constant of the encapsulant 2102 and buried compressible material 2104 disposed between the plates. When an external force is applied to the device adjacent the capacitor, the orthogonal component of the force deforms the compressible material (the deforming force) thus changing the capacitance, which is captured by the device and therefore can be used as a strain gauge. Thus, the embedded capacitor may be used as a capacitive strain gauge.

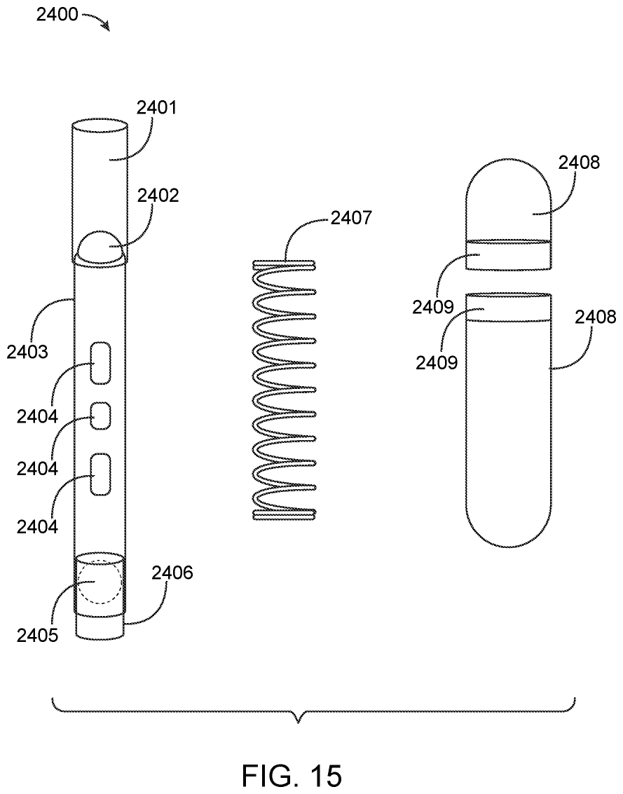

FIG. 15 shows an example of an unibody illumination system 2400. This example comprises the three components shown as depicted including light emitters 2402 and 2405. One light emitter 2402 is pointed axially and one light emitter 2405 points radially outward, and these are examples of possible directions for illumination targets, but it not intended to be limiting and the light emitters may emit light in any desired direction. The electronics are located on a printed circuit board or substrate 2403, and additional components 2404 can comprise integrated circuits (IC), sensors, and passive electronics components or any other component needed. Lightguide structures 2401 and 2406 adjacent light emitters 2402 and 2405, may be used to help to direct the light to a specified treatment area.

The device may also include a wireless coil receiver 2407 that is connected electrically to the circuit as well and may be wound around the substrate 2403. The entire structure may also be encased in a transparent hermetic shell 2408, with metal welding structures 2409 such as rings disposed around the device that may be soldered, welded or otherwise coupled together to create the desired seal.

The transparent outer shell 2408 can be made of glass and may not be required if the biocompatibility requirements of the application are met by the other materials. For example, certain electronics substrates 2403 may be biocompatible and transparent enough to allow light to pass through.

In some examples, a unibody illumination system may be operably coupled to one or more surgical tools. Such surgical tools include pre-existing, off-the-shelf tools or integrally constructed tools. The unitary encapsulated device may include one or more tool attachment points (not shown) on an external or tissue facing surface to allow the illumination system to be coupled to a device either fixedly or releasably coupled thereto. Examples of surgical tools are described in more detail below.

### Examples of Treatment with Encapsulated Devices

The encapsulated devices disclosed herein may function as an illumination system to treat various types of diseases including cancer and neurodegenerative disease. Neurodegenerative diseases are a group of conditions that cause the progressive loss of nerve cells in the brain and/or spinal cord. This damage can lead to a variety of symptoms, including memory loss, difficulty thinking or reduction of cognitive function, movement problems, and behavioral changes. There are over 50 known neurodegenerative diseases, some of the most common include Alzheimer's disease, Parkinson's disease, Huntington's disease, multiple sclerosis, Amyotrophic lateral sclerosis (ALS), Creutzfeldt-Jakob disease (CJD), Friedreich's ataxia, and Multiple system atrophy (MSA), dyskinesia.

Concerning the treatment of various cancers and other diseases, this includes but is not limited to Basal Cell Carcinoma (e.g. Basal Cell Epithelioma); Squamous Cell Carcinoma; Actinic (e.g. Solar) Keratosis; Skin Cancer; Solid Tumor; Prostate Cancer; Esophageal Cancer; Transitional Cell Carcinoma (Urothelial Cell Carcinoma); Bile Duct Cancer (e.g. Cholangiocarcinoma);

Endobronchial Cancer; Kidney Cancer (e.g. Renal Cell Cancer); Renal Cell Carcinoma; Choroidal Neovascularization; Brain Tumor; Glioma; Neurofibroma; Head And Neck Cancer; Hepatocellular Carcinoma; Metastatic Colorectal Cancer; Nasopharyngeal Cancer; Pancreatic Cancer; Benign Prostatic Hyperplasia; Age Related Macular Degeneration; Coronary Disease; Cutaneous Vascular Malformations; Peripheral Arterial Disease (PAD); Peripheral Vascular Disease (PVD); Mycosis Fungoides; Psoriasis; Glioblastoma Multiforme (e.g. GBM); Inflammatory Bowel Disease; Colorectal Cancer; Malignant Mesothelioma; Ovarian Cancer; Viral Infections; Colon Cancer; Graft Versus Host Disease (GVHD); Carcinomas; Sarcomas; Acne Vulgaris; Coronary Artery Disease (CAD) (e.g. Ischemic Heart Disease); Breast Cancer; Non-Small Cell Lung Cancer; Small-Cell Lung Cancer; and Bladder Cancer.

Concerning neurodegenerative diseases these illumination systems described herein may be used to provide deep brain stimulation in patients with these diseases such as Parkinson's disease, amyotrophic lateral sclerosis (ALS), Alzheimer's disease, or any other condition where neurostimulation is beneficial. One form of neuroprotective brain therapy is known as Photobiomodulation (PBM), which describes the use of red or near-infrared light to stimulate, heal, regenerate, and protect tissue that has either been injured, is degenerating, or else is at risk of dying. One of the organ systems of the human body that is most necessary to life, and whose optimum functioning is most worried about by humankind in general, is the brain. The brain suffers from many different disorders that can be classified into three broad groupings: traumatic events (stroke, traumatic brain injury, and global ischemia), degenerative diseases (dementia, Alzheimer's and Parkinson's), and psychiatric disorders (depression, anxiety, post-traumatic stress disorder). There is some evidence that all these seemingly diverse conditions can be beneficially affected by applying light to the head. There is evidence that supports that PBM could be used for cognitive enhancement in normal healthy people.

PBM is understood as a mechanism by which nonionizing optical radiation in the visible and near-infrared spectral range is absorbed by endogenous chromophores to elicit photophysical and photochemical events at various biological scales without eliciting thermal damage. It involves the use of nonionizing forms of light from sources, including lasers, light-emitting diodes (LEDs), and broadband light, in the visible and near-infrared spectrum, to cause physiological changes and therapeutic benefits.

Photobiomodulation treatment (PBMT) has been tested in preclinical models of neurodegenerative disease, including Parkinson's Disease, and is found to have the potential for therapeutic benefit. In both rodent and non-human primate models of Parkinson's disease, near-IR light was found to have a neuroprotective effect. In one mode of action, the light prevents the dopaminergic cells of the substantia nigra pars compacta (SNc) from dying, thus halting disease progression. Near-IR light therapy applied to animal brains was found to have no measurable toxicity even at higher doses and over long periods of time. The near-IR treatment is effective over a wide range of light intensity and dose parameters (known as a therapeutic window). A mechanism of the neuroprotective effect is that near IR light stimulates mitochondria by increasing adenosine triphosphate (ATP) content and electron transfer in the respiratory chain, through activation of photoacceptors (e.g. cytochrome oxidase), together with modulating reactive oxygen species and the induction of various transcription factors.

To investigate PBMT via an in vivo proof of concept experiment, wild-type and transgenic mice inoculated with one or more pathologies (for example, alpha synuclein (αS) pathology) underwent PBMT using an illumination system as described herein. It is notable that any progressive animal model, which has been shown to accelerate downstream development of Parkinsonian symptoms would be sufficient for such proof-of-concept PBMT study.

It would be expected that the neuroprotective effect caused by PBMT in vivo would be pronounced in many classes of neurodegenerative disease models, including (i) pharmacological (e.g., reserpine, 6-hydroxydopamine, and 1-methyl-4-phenyl-1,2,3,6-tetrahydropyridine); (ii) pesticide and herbicide (e.g., paraquat, rotenone, and trichloroethylene), and (iii) genetic (e.g., α-Synuclein, PARKIN, PINK 1 and DJ1, and LRRK2). Specifically, reserpine prevents the storage of monoamine neurotransmitters (including dopamine, norepinephrine, and serotonin) in neurons' presynaptic terminals. This causes depletion of these neurotransmitters in the striatum, resulting in motor symptoms that resemble PD (Parkinson's disease), including rigidity and slowness of movement. 6-hydroxydopamine (6-OHDA) is a neurotoxin with a similar chemical structure to dopamine. It can be taken up by dopamine transporters, which are expressed on the synapses of dopamine-producing neurons. 6-OHDA also has a high affinity for norepinephrine transporters, so it is often co-administered with a norepinephrine reuptake inhibitor in order to spare noradrenergic neurons. When 1-methyl-4-phenyl-1,2,3,6-tetrahydropyridine (MPTP) enters the brain, it is metabolized by astrocytes into a toxic metabolite called 1-methyl-4-phenylpyridinium (MPP+). It then gets taken up by dopaminergic neurons, where it induces toxicity via mitochondrial dysfunction. Many pesticide and herbicide models for PD function similarly to the pharmacological models, in that they bear structural similarity to dopamine. One example is paraquat, a common agricultural herbicide. Mice treated with paraquat show α-synuclein accumulation in the substantia nigra. Rotenone is a combination of herbicide and pesticide that, like MPP+, preferentially targets dopaminergic neurons in the nigrostriatal pathway and induces accumulation of α-synuclein. Its mechanism of neurotoxicity likely involves triggering the intracellular release of dopamine. Because it is not dependent on the dopamine transporter, rotenone also exerts some toxicity on other neurotransmitter systems, although dopaminergic neurons are its primary targets. Trichloroethylene (TCE) is an industrial degreasing agent that is recognized as an environmental risk factor for PD. Rats treated with TCE develop α-synuclein accumulation, loss of nigrostriatal dopaminergic neurons, and other PD hallmarks. However, they do not show significant depletion of dopamine.

While most cases of human PD have no known genetic origin, around 10% of PD cases are the result of a genetic mutation. These mutations, which have been localized to 14 different genes, provide insights for creating genetic models of PD. Mutations in the α-synuclein gene have been implicated in several cases of familial Parkinson's disease. While most PD patients do not possess these mutations, α-synuclein overexpression models can nonetheless be useful for investigating PD therapies. These models may use the mutations identified in familial PD or a transgene that causes overexpression of full-length α-synuclein or a truncated version. An important advantage of these models over the pharmacological and toxicological systems is that they exhibit progressive accumulation of α-synuclein with age. Numerous other genetic models of PD have been generated based on mutations observed in familial PD. These include mutations in PARKIN, a ubiquitin E3 ligase involved in mitochondrial homeostasis. PARKIN knockout mice have mild motor deficits that progress with age, as well as reduced dopamine release, α-synuclein pathology, and increased oxidative stress. However, they do not show loss of dopaminergic neurons in the nigrostriatal system. Similarly to PARKIN, recessive mutations in the PINK1 and DJ1 genes have been observed in familial PD. PINK1 and DJ1 knockout mice do not recapitulate PD pathology without additional insults. However, these animals show increased vulnerability to mitochondrial toxins. Dominant mutations in leucine rich repeat kinase 2 (LRRK2) have been observed in familial PD, and the gene also plays a role in sporadic Parkinson's disease. LRRK2 knockout or overexpressing mice do not show gross neurodegeneration, but they do display α-synuclein accumulation due to deficits in Golgi complex function and microtubule-based transport. These models also show enhanced pathological progression induced by α-synuclein overexpression.

In one approach, anesthetized disease models were exposed with red light at 660 nm at approximately 40 mW/cm² , for 90 seconds per session, twice per day for the duration of an experimental treatment cycle. The target duration of the total test cycle was several months, or until the appropriate behavioral demonstration of significant biological or morphological differences between controls (sham) and treatment groups. Positive control were transgenic mice without any exposure to PBMT treatments. Negative control were wild type mice (not transgenic) not expressing any mutant construct under the direction of a promoter. Any example of treatment parameters may include any of the wavelengths of light disclosed herein, illumination power of less than 1 mW or less than 5 mW or less than 10 mW, or from less than 1 mW up to 10 mW, and any of the power levels may be used for any desired duration or duty cycle. In any example, up to 125 Joules total power may be delivered to the substantia nigra area of the brain. In any example, light at any power or power density may be delivered for 5 seconds on and then 60 seconds off, repeated continuously for 25 days. In any example, the power may be delivered for 10, 15, 20, 25, 30, 35, 40, 45, 50, 55, or 60 seconds and an optional off period may be 5, 10, 15, 20, 25, 30, 35, 40, 45, 50, 55, or 60 seconds before the illumination is turned back on again. The illumination in any example may be provided for 1, 2, 3, 4, 5, 6, 7, 8, 9, 10, 11, 12, 13, 14, 15, 16, 17, 18, 19, 20, 21, 22, 23, 24, 25 or more than 25 days as needed. Therefore, any permutation or combination of the operating parameters disclosed above may be used to treat a patient.

Studies have been conducted using various treatment paradigms using both neurotoxin-based and transgenic lines of rodent models of disease. One example of transgenic mice expresses the mutant human alpha-synuclein under the direction of the mouse protein promoter. These mice may have been found to be useful in studying human neuronal alpha-synucleinopathies, such as familial Parkinson's Disease. Certain of these transgenic mice express a human variant alpha-synuclein (full-length, 140 amino acid isoform) under the direction of the mouse protein promoter.

It is understood that mice homozygous for certain of the transgenic insert are viable, fertile and normal in size. At eight months of age, some homozygous mice develop a progressively severe motor phenotype. Presentation of the phenotype may manifest at 14-15 months of age (on average). Lax grooming, weight loss and diminished mobility precede movement impairment, partial limb paralysis, trembling and inability to stand. It has been seen that immunohistochemistry analysis of mutants between eight to twelve months of age reveals widely distributed alpha-synuclein inclusions, with dense accumulation in the spinal cord, brainstem, cerebellum and thalamus. The appearance of alpha-synuclein aggregate inclusions parallels the onset of the motor impairment phenotype. Axons and myelin sheaths exhibit progressive ultrastructural degeneration. Immunoelectron microscopy and biochemical analysis show the inclusions in neurons are comprised primarily of 10-16 nm fibrils of alpha-synuclein. It has been demonstrated that the structure, location and onset of the inclusions seen in the mutant mice resemble characteristics seen in human neuronal alpha-synucleinopathies, such as familial Parkinson's Disease. Additionally, these mice exhibit impaired odor discrimination and detection beginning at 6 months of age. Homozygous mice have a high incidence of nonproductive mating. Aggression is observed, particularly in males, and may be due at least in part to the aspects of the genetic background.

To date, a number of investigators have examined the neuroprotective effects of PBMT in vivo in Parkinson's models of disease. However, these studies used any manner of external light illumination to effect PBMT in these models. Instead here, it is demonstrated that the neuroprotective effect of implant-based illumination enhances PBMT causing significant improved outcomes. Any of the implantable monolithic illumination systems described herein is capable of achieving pulsed, chronic, cyclic near-infrared illumination of the central nervous system at any wavelength. As stated above, PBM works on the principle that light-sensitive molecules in the body known as chromophores are excited by photonic stimulation. It is now understood that hemoglobin, myoglobin, and COX are the only 3 chromophores in mammalian tissue capable of absorbing light in the NIR range (600-900 nm wavelength). The implantable monolithic illumination systems described herein may be programmed with a Light Revolution to modulate and enhance PBMT. The Light Revolution may be optimized to fit one or more treatment parameters and/or paradigms. The Light Revolution may be thought of as a programmed course or series of pulsed, chronic, cyclic to optimize patient outcomes via enhanced neuroprotection to halt disease progression. Pulse means periodic alternating modulation of light stimulation over a range of wavelengths. Chronic means continuing, persistent or otherwise long-lasting light stimulation. Cyclic means a sequence of a recurring and successive light stimulation. A Light Revolution may comprise any or all of the known treatment parameters. In one example, a Light Revolution is pre-programmed. In some examples, a Light Revolution is modified in response to patient response.

While various theories and mechanisms of action have been described above, one of skill in the art will appreciate that these are not intended to be limiting and the examples of devices and methods disclosed herein are not necessarily intended to be bound by these theories or mechanisms of operation.

### Surgery and Implantation

One approach to implant any of the illumination systems described herein may be to employ a surgical procedure, which creates an opening in the skull for a portion or the entirety of the implanted device to be placed into the skull. A portion of the device that emits light will be implanted close to the substantia nigra pars compacta in the midbrain or any desired target treatment tissue. A portion of the device may reside on the outside of the skull but under the scalp, similar to a cochlear implant.

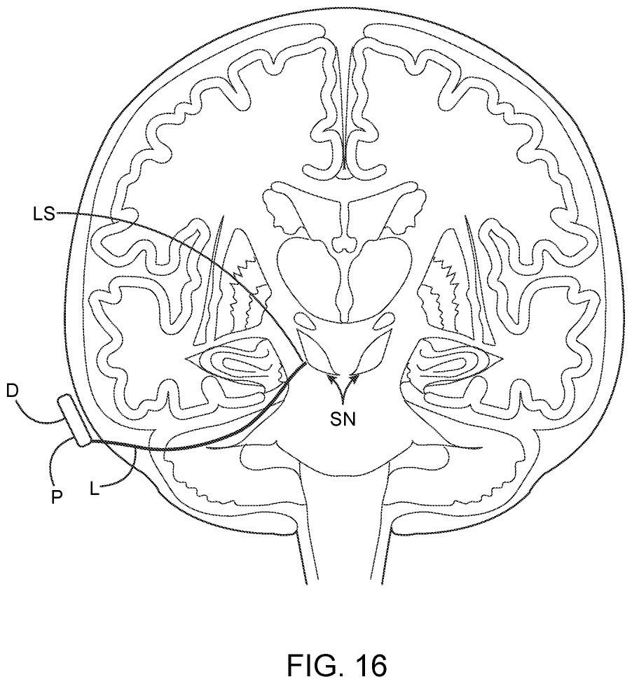

FIG. 16 shows one way in which such a device D may be implanted adjacent the substantia nigra pars compacta SN tissue in the brain. The device may be any of those disclosed herein or another. The dark line represents the path of an implanted device lead L, which would contain a light source LS at the distal tip. A portion P of the device, for example containing electronics or a wireless coil, may be located on the outer surface of the skull. If necessary, bilateral devices can be placed from either side, but a single device may sufficiently illuminate both substantia nigra pars compacta SN or it may be therapeutically unnecessary to treat both.

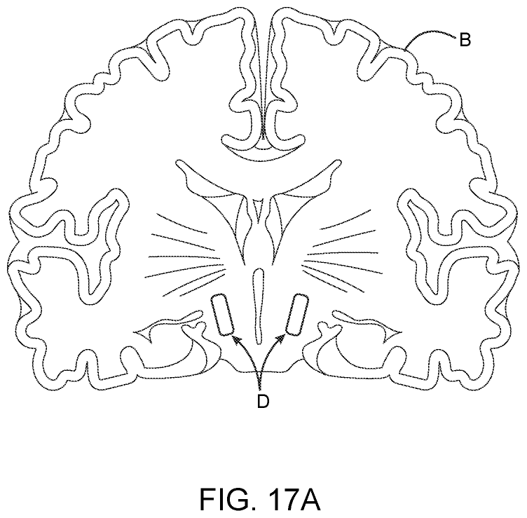

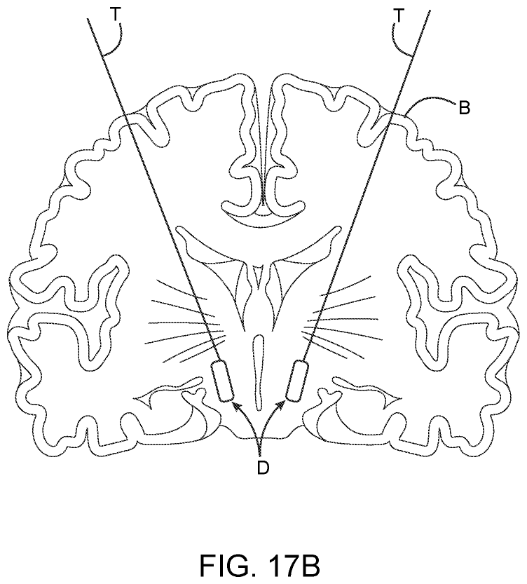

FIG. 17A shows another approach to a treatment procedure, where one, or two, or any number of smaller, fully enclosed devices D are delivered with a biopsy needle directly to the midbrain area adjacent the target treatment area such as the substantia nigra pars compacta of the brain B. This follows a similar delivery path to deep brain stimulator electrodes, but these devices would deliver light for PBM therapy. The device may be any of those disclosed herein or another device.

FIG. 17B shows yet another approach to a treatment procedure where one or two, or any number of smaller, fully enclosed devices D are delivered from the top of the head along a treatment path T to the target treatment area, essentially mimicking how surgeons introduce DBS device probes today. The devices may be delivered with a biopsy needle directly to the midbrain area adjacent the target treatment area such as the substantia nigra pars compacta of the brain B. This follows a similar delivery path to deep brain stimulator electrodes, but these devices would deliver light for PBM therapy. The device may be any of those disclosed herein or another device.

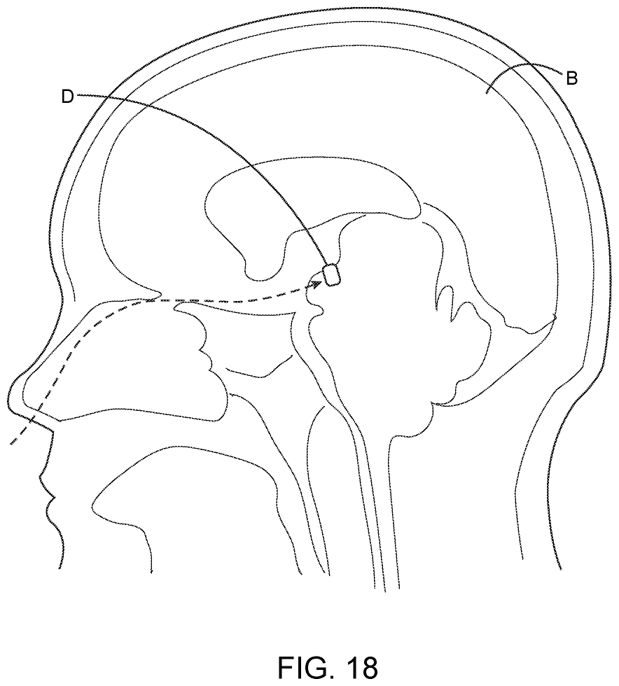

FIG. 18 shows still another approach to a treatment procedure, using an intranasal delivery route. Here, a device D which may be any of those disclosed herein or another device is delivered through a nostril intranasally into the brain B adjacent the target treatment tissue, as shown by the dotted line. Thus, the same result is achieved as described above but changing the surgical approach. The device may be placed with endoscopic and/or image guidance.

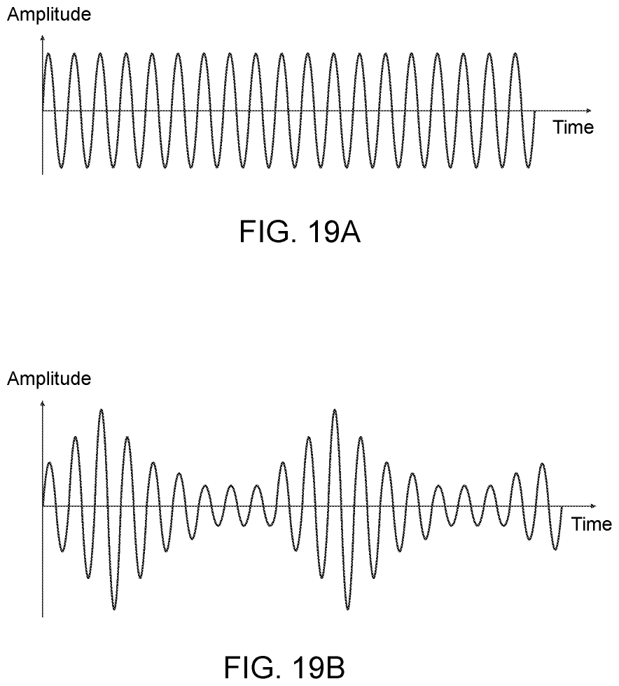

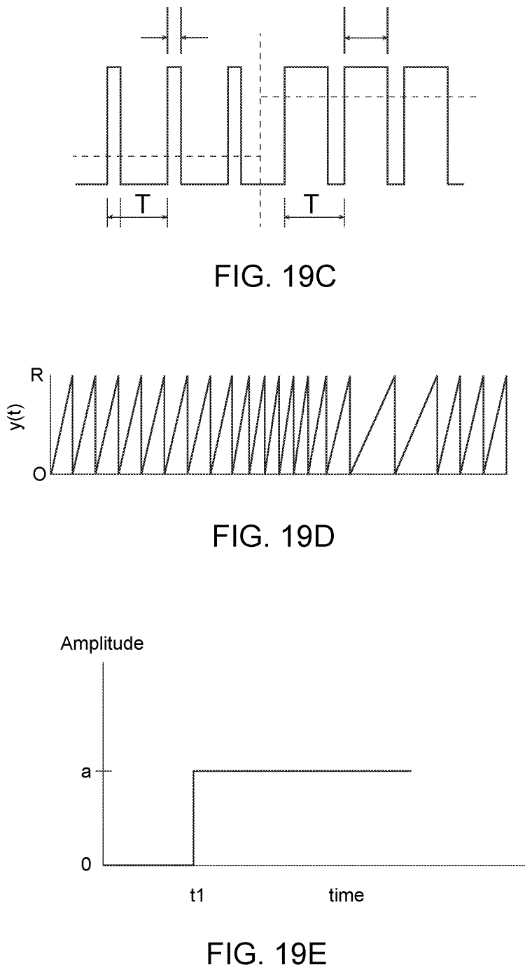

FIGS. 19A-19E show examples of different waveforms of light intensity that may be used to treat a patient for any condition, including those described herein. The primary mechanism of therapeutic action is through interaction with light and the cells in the brain. The intensity of the light may be modulated, in the same way that electrical signals are modulated for neurostimulation of electrical pathways.

FIG. 19A shows light delivered in a constant amplitude sine wave waveform.

FIG. 19B shows light delivered in an amplitude-modulated sine wave.

FIG. 19C shows light delivered in pulses of fixed or varying duty cycle (width) and frequency. Pulse width modulation of the waveform may include narrow pulses and wider pulses where the width of the pulse is the time the light is delivered. The amplitude of the pulses may be the same or different.

FIG. 19D shows light delivered in pulses of sawtooth/triangular waves. The amplitude may be the same or vary.

FIG. 19E shows light is constantly delivered and always on at a constant amplitude as long as the illumination device is powered on (at time=t₁).

Additional parameters that can change include total duration of each session, the repetition rate of the sessions, and the overall duration of treatment. Other variations on these themes include pulse frequency modulation.

A primary mechanism of therapeutic action is through interaction with light and the cells in the brain, so dosage refers to light intensity and time.

Treatment includes brief treatment sessions performed over time on a regular basis. This may include treatment with light immediately after a tumor has been resected, or the initial treatment may be delayed for a period of time after tumor resection. The initial treatment may be performed intraoperatively, or after the surgical wound has been closed. Another treatment may include initial treatment with light illumination intraoperatively and then immediately after surgical closing. During each session, an external transmitter device will be provided to the patient and worn in a manner analogous to a hat, cap, beanie, or audio headset. That transmitter will contain a power source and wireless transmitter device that can send power as well as bidirectional data to the implanted device. The device will be used either in an outpatient setting, or more likely, at the patient's home regularly. Possible examples are: 30 minutes per day each morning; 1 hour per session, 3 days per week; or some combination thereof.

In other examples of non-invasive PBM treatments for PD, there are a few groups that have examined its efficacy in human subjects. Hamilton et al. (2019) and Santos et al. (2019) have recently reported slight improvements in motor and non-motor symptoms in PD patients after the administration of PBM using extracranial sources. In their trial, Santos et al. (2019) delivered PBM by alternating a 670 nm LED between the subjects' left and right temples in six 1-min blocks per session. Subjects were treated twice a week for a total of nine weeks and demonstrated significant improvements in motor scores (Santos et al., 2019).

The external power supply of the device will be plugged into the AC mains power during use, and/or contain a rechargeable battery so it is portable. The device and its usage will be minimally intrusive into the patient's lifestyle. The device may be connected to the Internet directly, or through the patient's smartphone, so that clinicians can monitor the progress.

With regard to the total duration under treatment, it is expected that the patient will continue to self-administer the therapy for months or years in order to see a lasting benefit in disease modification.

A burr hole is an access point to the brain, made as a part of most brain surgeries. A 1-2 cm diameter burr hole is drilled out of the patient's skull in order to access the brain. At the end of the surgery, the hole can be covered with a plate of metal or other material, which can be affixed to the skull using screws. Depending on the situation, the hole may not need to be covered or filled, at the discretion of the surgeon.

The examples disclosed herein include description of a long, thin light-emitting device that is designed to emit light in the axial direction and also may emit light in the radial directions. This requires that the light-emitting device be constructed with an external encapsulation material that is both biocompatible and also transparent or partially transparent (semitransparent, translucent) to the wavelength(s) of light emitted, or that has any other desired optical properties. The encapsulation material can be made of various materials including ceramic, such as glass, a crystalline material such as sapphire, or a polymer material, or any other encapsulant material disclosed herein or otherwise known in the art. The other purpose of the encapsulation is to segregate biological fluids and tissues from the internal components of the device, for device reliability and also biocompatibility reasons.

The encapsulation can have transparent and non-transparent portions. Non-transparent portions may be used for hermetic sealing purposes, for example titanium pieces that can be welded together. Non-transparent portions may also include coiled wire, for receiving wireless power and data from external sources. For optimal performance, at least one end of the light-emitting device will be made of transparent/semi-transparent materials.

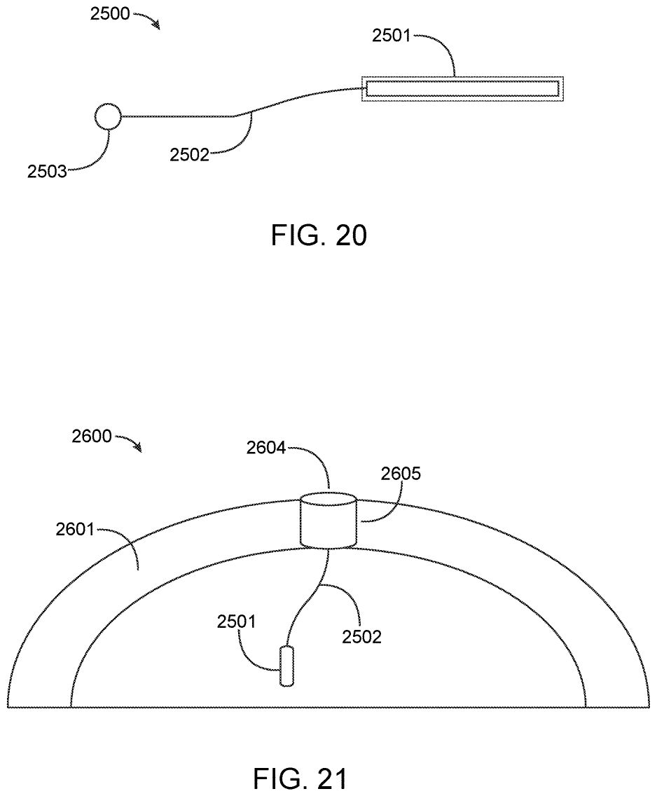

FIG. 20 shows an example of an implantable illumination device 2500 that may be used for any of the treatments disclosed herein. It may have any length but may be long in order to ensure that the light is delivered into deep locations in the body. Therefore, this example may be colloquially as referred to as a tea bag where there is a proximal portion that is disposed either outside the patient or adjacent the burr hole, and a distal portion that dangles and can be disposed in a target treatment area away from the proximal portion. A tether couples the proximal and distal portions together and may also serve as the electrical and/or mechanical connection between the proximal and distal portions. For the purposes of device explanation and also fixation within the brain tissue (or other tissue), a thread or wire 2502 can be attached to one end of the light-emitting device 2501 that is encapsulated. This thread 2502 can be stiffer or more flexible depending on whether that quality is needed to keep the device in place. The material can be polymer-based; a fiber such as aramid, nylon, silk, polyester, or polypropylene; or a metal such as gold, platinum, nitinol, or stainless steel. This tether can be encapsulated in a polymer-based tube or overmolded. The figure also shows a securement component 2503, which can be attached to the skull using screws, adhesive, or another method to keep the implanted device in a fixed location. Other electronics may be disposed in the securement component 2503 or they may be coupled thereto as discussed below.

The tether wire 2502 can serve several purposes. First, the tether can be designed to remain in a state of tension after installation, holding the light-emitting device in place. In that case, the tether will be attached to a burr hole cap, directly attached to the skull via a securement adapter plate (tether wire holder), or some combination thereof. The length of the tether can be adjusted with precision by the surgeon during the surgery, and will be secured in place afterward.

The second purpose of the tether wire 2502 is to be an electrical connection between two components of the device. If there is an electronic component sitting on or within the skull, the tether wire can contain a powering and/or communication link between the burr hole cap and the light-emitting device. The wire may be a bundle of wires, or a cable assembly, containing multiple conductors separated by insulator materials. The tether wire may also act as a reference, or ground, electrical connection to aid sensors or stimulation functionality within the device.

A third potential purpose of the tether wire 2502 is to act as an antenna element, receiving or sending power and data between the implanted device and some external device situated outside the body. The antenna element must be made of a conductive material (e.g. a metal), and its length will be determined by the frequency or frequency range of interest. The antenna portion may comprise only a portion of the tether wire, and the conductive antenna may be co-located with a non-conductive tether as part of a cable assembly. In this case, only a portion of the tether wire 2502 would be made of a conductive material to act as an electrical antenna.

FIG. 21 shows implantation of an illumination device 2600. This example describes the implantation of a medical device component, the burr cap 2604, that can fit within a drilled hole 2605 in the skull 2601. The top of the device can sit approximately flush with the original contour of the skull, meaning that the patient would not have any additional bumps on the head, and that the device is partially protected against damage from falls and other external head trauma. The illumination source with light-emitting portion 2501 of the device 2600 would still be located at the brain tissue of interest for illumination, and the devices would be connected by a tether wire 2502 to the burr cap. The burr cap 2604 may be a passive device component made of polymer, ceramic, metal, or some combination thereof, or the burr cap 2604 may contain active electronic sub-components, as described elsewhere in this specification.

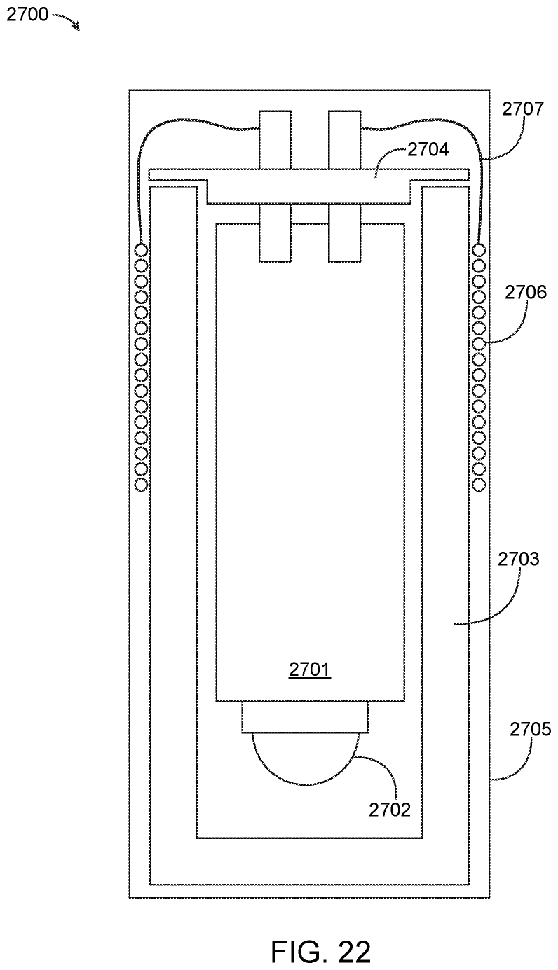

FIG. 22 shows an example of a light-emitting device 2700 that is an encapsulated component with a light source 2702 such as a light-emitting diode (LED), laser, or other light-emitting photoelectric source contained inside a biocompatible encapsulant 2703 which may be any of the materials disclosed herein. The light-emitting component 2702 may emit light in the axial or radial direction, or there may be multiple sources 2702 within the same encapsulant 2703. A printed circuit board 2701 provides mechanical stability and electrical connection to a feedthrough 2704 that passes through the encapsulant, which allows the device to have electrical connections outside the hermetic seal. Wires 2707 and an electrical coil/antenna 2706 receive and provide power and/or data to or from the device. Elements 2706 and/or 2707 may be coils in the case of near-field powering, or another shape such as a wire antenna in the case of far-field RF powering. An outer coating 2705, which could be a transparent polymer such as silicone or polyurethane, can be optionally added for mechanical protection or improved biocompatibility, or to provide additional optical properties for controlling the light emitted.

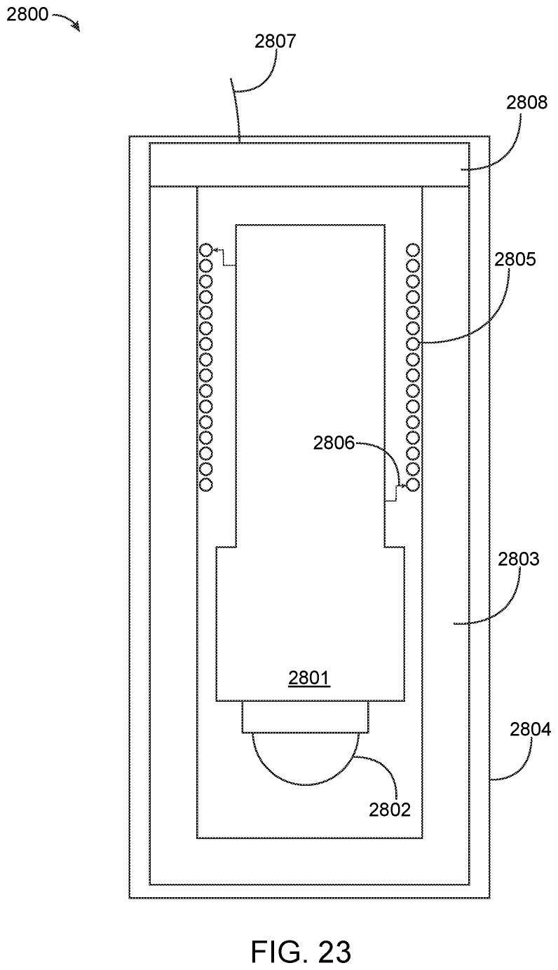

FIG. 23 shows an alternate example of a light-emitting device 2800, which has a near-field antenna coil 2805 located within the encapsulant 2803. This means that there would be no electrical connections required between the inside and outside of the encapsulant 2803. A printed circuit board 2801 provides mechanical stability and electrical connections for all the internal components, including an LED light source 2802. Wires 2806 provide an electrical connection to the coil 2805. An outer coating 2804, which could be a transparent polymer such as silicone or polyurethane, can be optionally added for mechanical protection or improved biocompatibility or for additional optical properties for controlling the emitted light.

A passive tether wire 2807 is shown, connected to the encapsulation, which provides mechanical attachment for the light-emitting device (such as coupling to the burr cap or directly to the skull). Also shown is a lid 2808 to the encapsulant, which could be of a different material than the rest of the encapsulation 2803, and may be welded, brazed, or otherwise sealed to the encapsulation material 2803. The size of this device and any device disclosed herein may be any size to fit the intended use and space available. In this example, the device may be cylindrically shaped with a diameter that may be less than 5 mm, or less than 4 mm, or less than 3 mm, or less than 2 mm, or less than 1.5 mm, or less than 1 mm.

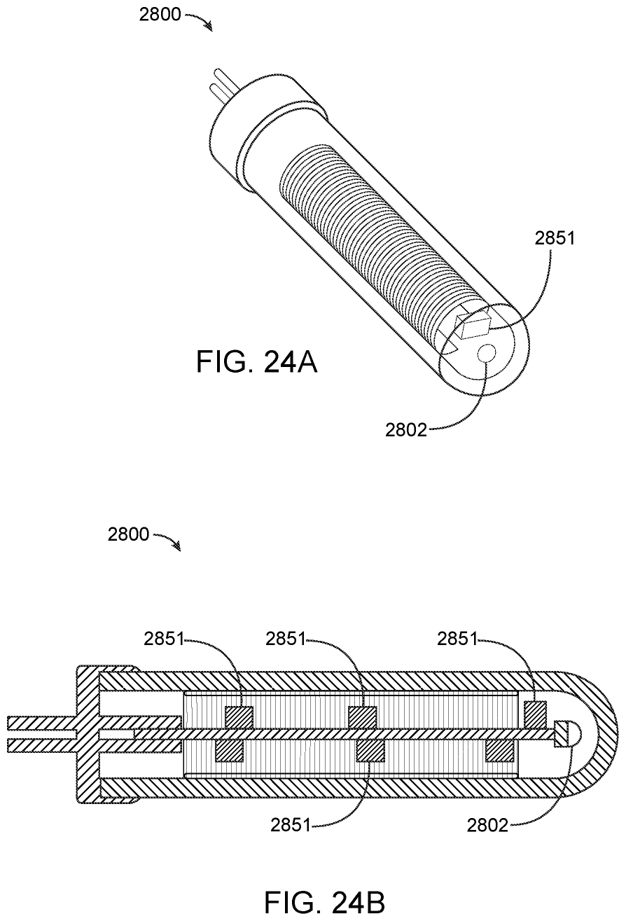

FIGS. 24A-24B illustrate additional details of device 2800 in FIG. 23 above. FIG. 24A shows a perspective view of the device 2800 and FIG. 24B shows a cross-section of FIG. 24A taken along the longitudinal axis of the device. Shown are electronics components 2851 (e.g., resistors, capacitors, inductors, diodes, integrated circuits, etc.) that may be located on the printed circuit board, along with the LED 2802 at the distal end of the printed circuit board.

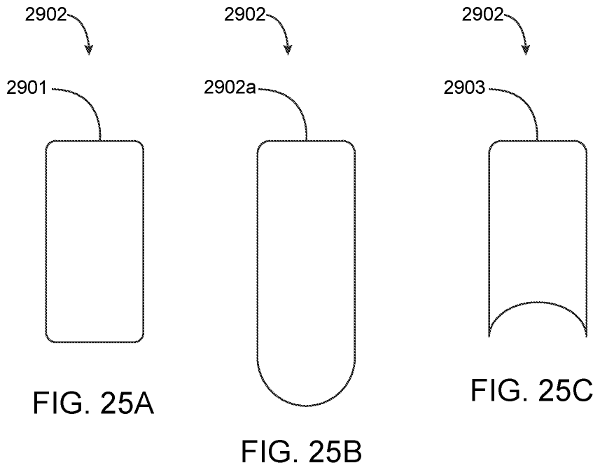

FIGS. 25A-25C show examples of devices with distal shapes to control light distribution. Assuming that the light is designed to radiate from the distal axial end of the light-emitting device 2902, the distal end of the device can be shaped to control the distribution of the light. Shown are examples of different shapes.

FIG. 25A shows a flat lens 2901 which helps ensure that the emitted light is substantially parallel with the longitudinal axis of the device, or that the light is emitted orthogonally to the surface of the flat lens. The flat lens will minimally impact the angle of such light emitted from the light source, which could be well-suited for collimated light such as from a laser-based source.

FIG. 25B shows a convex lens 2902 which may be used to refract light inward toward a focal point. Light rays passing through the edges of a convex lens are bent most, whereas light passing through the lens's center remain straight.

FIG. 25C shows a concave lens 2903 which causes the light to diverge.

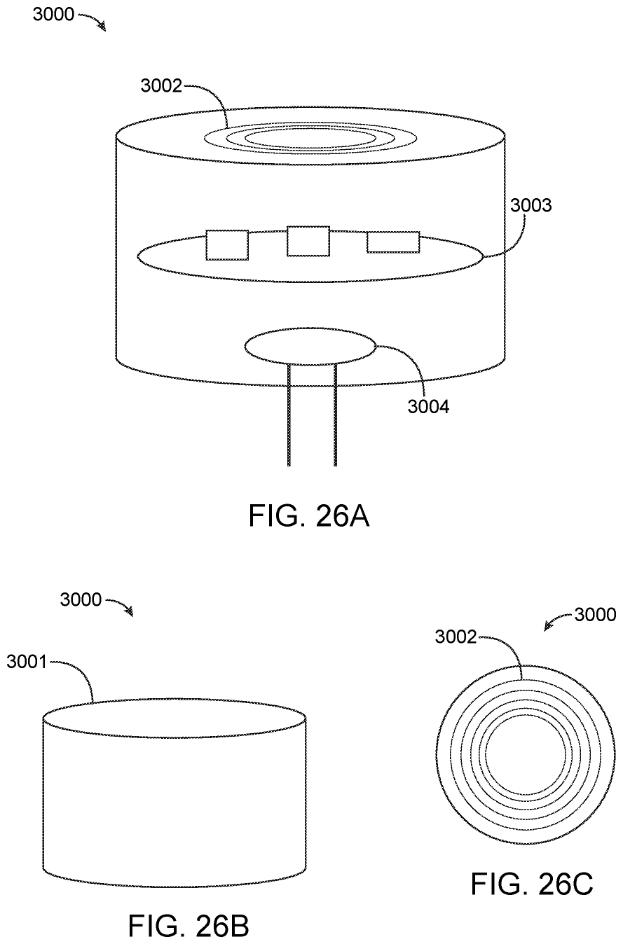

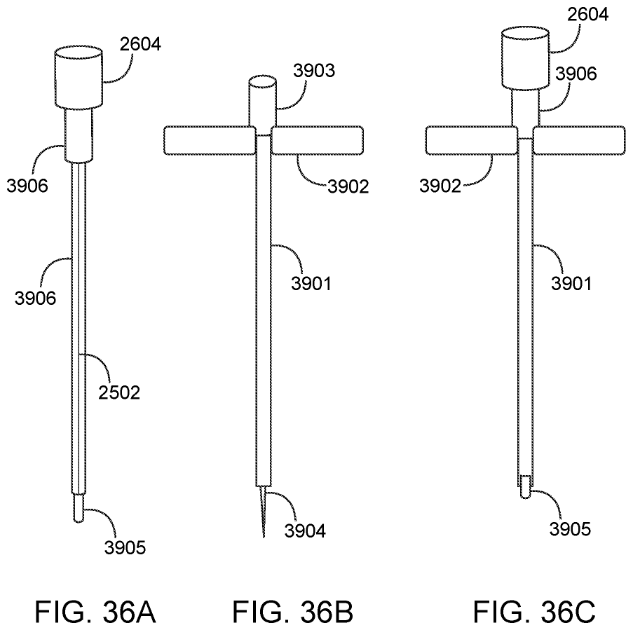

FIGS. 26A-26C show an example of a burr cap 3000 that may be used with any of the other devices (e.g. illumination devices) and/or methods described herein. FIG. 26A shows the burr cap 3000 with some optional components disposed therein while FIG. 26B shows an example of the exterior surface of the burr cap. FIG. 26C shows an example of a top view of the burr cap. The burr cap may have a cylindrically shaped outer surface 3001 (best seen in FIGS. 26A-26B) and may be hollow on the inside, and contain various electromechanical components (best seen in FIG. 26A). For example, a wireless power receiver coil 3002 that may act as an antenna may be disposed on a top surface of the burr cap and may be made of conductive material such as a conductive wire or potentially printed on or within the outer case of the cap. The cap may also contain a printed circuit board 3003, onto which can be mounted various components (e.g., resistors, capacitors, inductors, diodes, integrated circuits, etc.). A functional block diagram can be found in FIG. 36 of the components that may be disposed in the burr cap. As the burr cap may be hermetically sealed to prevent the human body from interacting with the components in the burr cap, there may also be an electronic feedthrough 3004 with one or two or more conductors (e.g. wire leads or pins) to transfer power and signals to and from the light-emitting device.

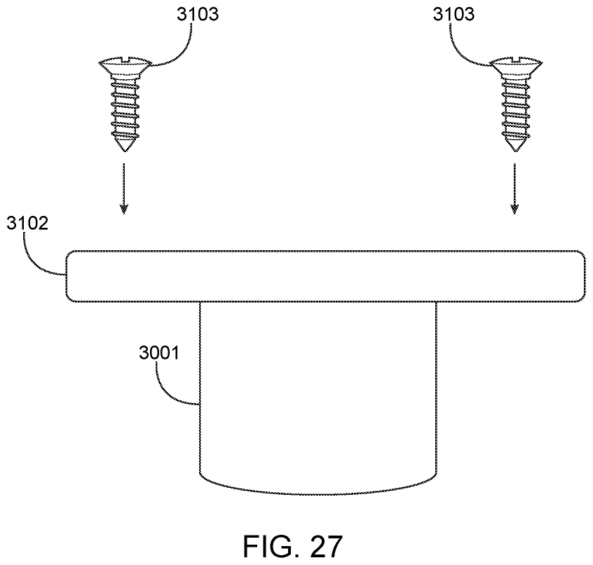

FIG. 27 shows the burr cap 3001 that may be positioned in a patient so that it sits inside a surgically drilled hole in the skull (skull not shown). To secure the cap, a larger-diameter circular plate 3102 may be attached to or integrated as a part of the cylindrically shaped burr cap, and the plate extends outside of the area of the drilled hole in the skull. This keeps the burr cap in place, and can also serve to protect the burr cap from mechanical damage after installation. One or more bone screws 3103 can be installed to further secure the burr cap to the skull. The plate 3102 can be made of a stiff material such as titanium or any other metal or material known in the art, which can act as a protective barrier.

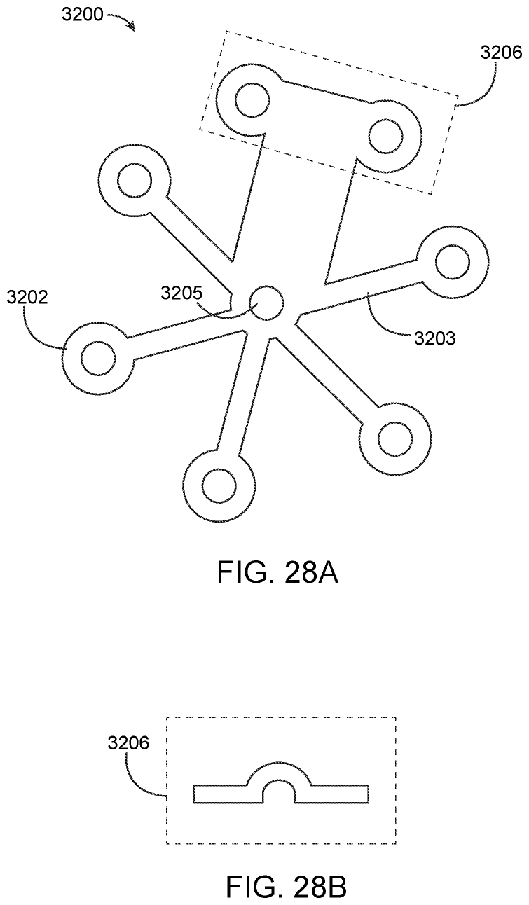

FIGS. 28A-28B show the burr hole (not illustrated) may be mated with a securement plate 3200 to close the burr hole and/or hold the implant to the skull or hold the burr cap. FIG. 28A shows a top view of the securement plate, and FIG. 28B shows an end view of one of the arms radiating out from the securement plate. The plate can contain grommets 3202 in through holes in the arms for bone screws, and arms 3203 that extend outward from the main body. It can also contain grommets 3205 for securing the plate to the burr cap. The securement plate may also contain conductive materials arranged as an antenna or coil, for the purposes of receiving power and bidirectional communication to an external device situated outside of the body. The securement plate can contain mechanical features such as a grooved plate 3206 for holding a cable/wire in place securely when the cable/wire is disposed in the channel and the securement plate is coupled to the skull. The grooved plate may be one of the arms radiating out from the plate. The securement plate may also be used to hold the tether wire in tension, so that the light-emitting device does not move further into the brain after the initial implantation surgery.

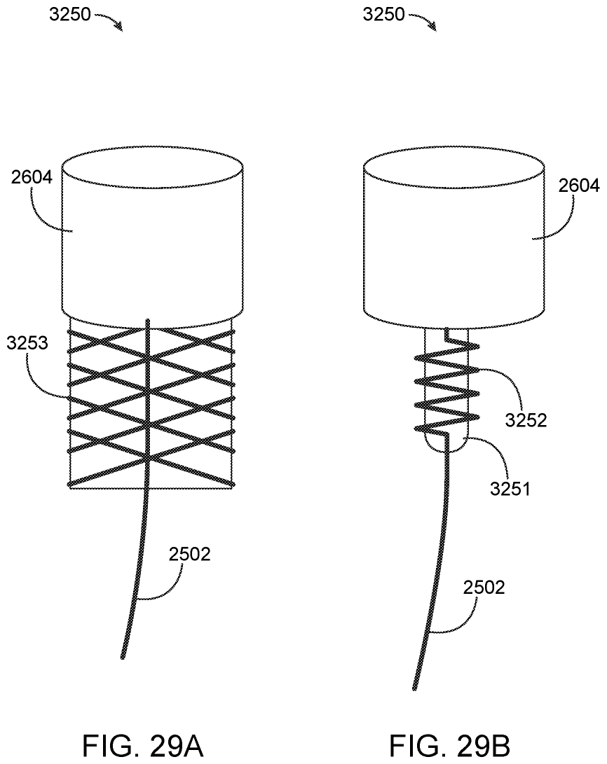

FIGS. 29A-29B show alternative methods of cable tensioning. Here, the device 3250 includes an element that can take up excess wire in the design of the burr cap itself. For example, in FIG. 29A the device includes a scissoring/accordion component 3253 that extends or shortens to control the length of the wire 2502 extending out of the burr cap 2604.

FIG. 29B shows an example of a device 3250 where the cable tensioning element may be a rotating screw/spool 3251. When turned or actuated by a surgeon, this can lengthen and shorten the wire coupled to burr cap 2604 into a coil 3252. The tether wire 2502 will thus be taut, and hold the light-emitting device in place after the surgery. These cable management tools shown may also be used to simply manage excess wire, while not holding the wire 2502 under tension.

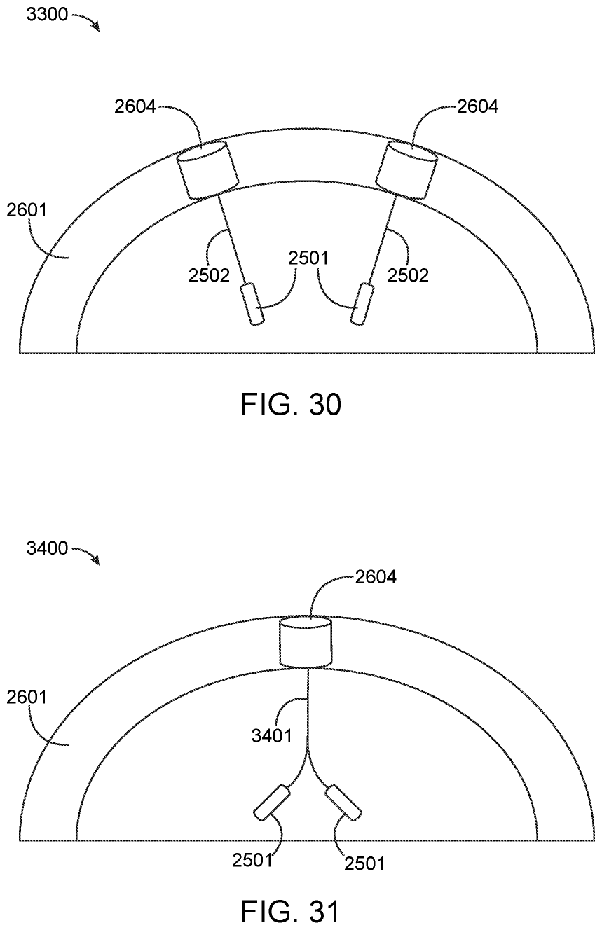

FIG. 30 shows that some anatomical structures in the brain are bilateral, for example the substantia nigra pars compacta (previously illustrated in FIG. 16 above) which is one of the key areas of neurodegeneration in Parkinson's Disease. Therefore, it may be advantageous to install devices 2501 on both sides of the brain. In that case, one option is to drill two holes through the skull 2601 and install two separate devices in the skull 2601. Shown are the light-emitting devices 2501, tether wires 2502, and burr hole caps 2604. The implanted device may be any of those disclosed herein. This of course is more invasive due to the two burr holes and therefore it may be preferred to form only a single burr hole.

FIG. 31 shows another example of an illumination device 3400 for illuminating multiple areas of the brain is to have multiple tethered light-emitting devices 2501 connected via tether wire 3401 to a single burr cap unit 2604, which is mounted in the skull 2601. The tether wire can have multiple connections, and can also contain a rigid or semi-rigid “Y” fork which is designed to orient the light-emitting devices in opposite directions. An introducer tool can hold the forks in-line as the device is introduced, and then they can spread out as they're inserted into the correct area of the brain. A single burr hole is less invasive than multiple burr holes.

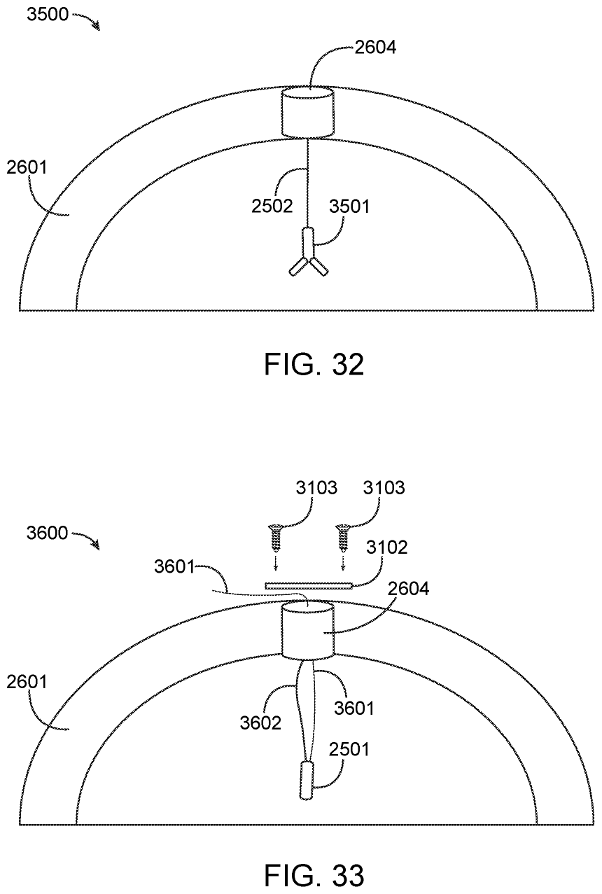

FIG. 32 shows another example of a device 3500 for illuminating multiple areas of the brain and may include lightguide structures as part of the light-emitting device 3501. Multiple “prongs” or protrusions can extend from the main body of the device, and these prongs can be made of polymer, glass, or another light-transmitting material. The prongs may be semi-rigid, so that the device can be introduced with the prongs held in an inline orientation (e.g. collapsed) until they reach the target area of brain tissue where they can then expand outward, here forming a Y-shaped illumination source. The prongs may direct light to desired areas of tissue to be treated. A single tether wire 2502 can connect the device to the burr cap 2604 disposed in the skull 2601. These lightguide elements can be made of a stiffer material such as high-durometer silicone or polyurethane, so long as the optical index of refraction of the material is different than that of brain tissue.

FIG. 33 shows a device 3600 with a hybrid tether wire, comprising a non-conducting cable 3601 and a conducting wire or multi-wire cable 3602, connecting the light-emitting device 2501 and the burr cap 2604 disposed in the skull 2601. The purpose of the non-conducting cable is to hold the light-emitting device in a stable position, and that can be a separate function from the conducting of electrical power and signals, handled by the conducting cable 3602. The burr cap 2604 may be secured to the skull 2601 with a plate 3102 that is attached to the skull with fixtures 3103 such as screws. An external connector wire or wires 3601 may extend out of the burr cap to allow other components or devices to be mechanically or electrically coupled with the implanted device.

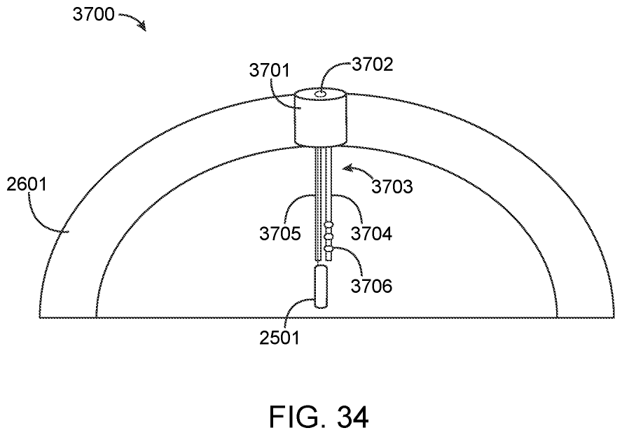

FIG. 34 shows an example of a device 3700 where the tether assembly 3703 between the burr cap 3701 inserted in skull 2601 and the light-emitting device 2501 can also have a distinct additional lumen 3704, running down the length of the tether, that may be used for drug delivery. A drug can be introduced at one end of the device, for example in a port 3702 that is integrated into the burr hole cap 3701, and the drug can then flow down the lumen to the same tissue area as the light-emitting device location. The lumen can contain holes 3706 along its length to allow the drug to diffuse into other areas of the tissue as well. The lumen may be coated with other chemicals during manufacture, which would prevent clogging due to fibrosis/thrombosis and also lubricate the tube to ease flow of the drug or to ease insertion of the tube into the tissue. Another portion 3705 of the tether 3703 may be used to couple the light device 2501 to the burr cap 3701 and may also be used to deliver power to the light device 2501 from the burr cap or external to the skull 2601. The portion 3705 may also be used to send or receive data to and from the light emitting device.

The input port for drug delivery may include a self-closing/self-healing membrane or a one-way valve design, which allows the drug to flow when introduced via percutaneous injection needle, but closes when such a needle is absent. This example allows concurrent treatment of tissue with light and a therapeutic agent, or a light activated agent may be delivered via the lumen and then activated by the light at the treatment site. During the implantation procedure excess wire in the tether can be controlled by using a securement plate such as element 3102 in FIG. 33 to secure the wire at the right length for the patient.

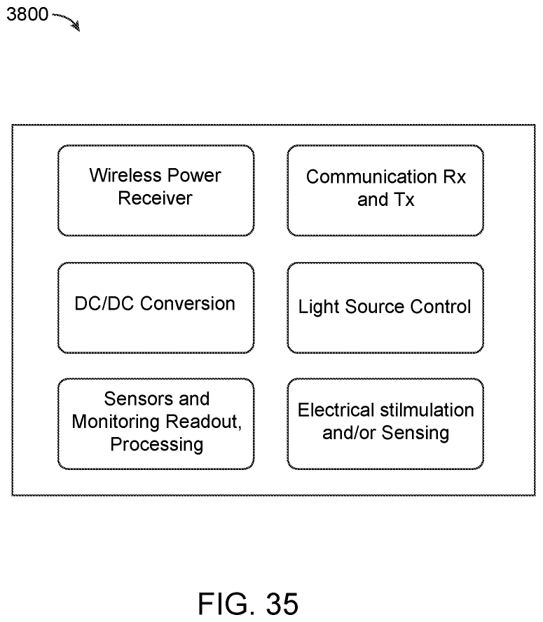

FIG. 35 shows a block diagram 3800 for the electronics which may be used in any example of an encapsulated illumination device disclosed herein. Either the burr cap or the light-emitting device may contain electronics and an antenna or coil that receives wireless power and enables uni-directional or bi-directional communication between the implanted device and an external device. The electronics features contained can include power electronics, communication with the non-implanted device(s), control electronics for the light source, and also readout electronics for sensors. Other components which may be used are also shown in FIG. 35.

FIGS. 36A-36C show examples of surgical instruments that may be used to help implant any of the devices disclosed herein.

Brain-implanted devices often require introducer tools to aid the surgeon. The surgeon will first drill a burr hole in the area where the device is to be inserted (the surgical approach is planned out in advance). Introducer tools may be designed similar to the examples shown in the figures.

FIG. 36A shows a light emitting device 3905 with tether 2502 pre-inserted into a pushing tool 3906. A pushing tool 3906 may be used to hold the device in place during insertion. The pushing tool is designed to provide rigidity in the axial direction, but may be removed after the light-emitting device 3905 is implanted. The burr cap 2604 may be coupled to the proximal portion of the light emitting device.

FIG. 36B shows a dilator 3904 with dilator handle 3903, for creating an entry port for the device through the tissue. A split sheath 3901 with handles 3902 is disposed over the dilator and allows the outer portion of the introducer to be removed after the device is implanted. The split sheath 3901, dilator 3904, and/or pushing tool 3906 can be designed with visible or radiopaque markings that, alongside imaging tools, allow the surgeon to precisely locate the device.

FIG. 36C shows the burr cap 2604, pusher 3906 and light emitting device 3905 coupled to or disposed in the split sheath 3901 with dilator 3904 removed. The burr cap remains outside of the split sheath.

Therefore, in use, the split sheath 3901 with dilator 3904 are inserted into the target treatment area to create a path for the device which may be any of the devices disclosed herein. Then the dilator is removed using the dilator handle 3903, and the pushing tool 3906 is advanced and helps to insert the light-emitting device 3905 to the precise anatomical target location. At the end of the insertion, the surgeon will remove the split sheath 3901 using handles 3902. Splaying the handles will split the sheath, usually along perforations allowing the sheath to be removed from the implant device. Then the surgeon will secure the burr cap 2604 and remove excess tether wire slack as described in other sections.

Additionally, in any example, the device may include a radially expandable member disposed around the device, such as a balloon. The balloon may be expanded after the device is implanted in tissue and helps to support the surrounding tissue (e.g. tissue walls in a tumor cavity) so that remaining tissue does not fold in or otherwise collapse on itself, thereby preventing illumination by the device. Therefore, the radially expandable member helps to ensure that tissue is supported and can be illuminated by the device. The balloon may be latex, silicone, polyurethane, or any other material known in the art. The balloon may be compliant or non-compliant.

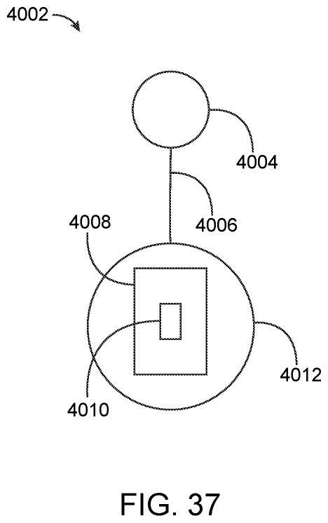

FIG. 37 shows an example of a device 4002 having an illumination source 4010 and a radially expandable member 4012 such as a balloon. The illumination source 4010 is encapsulated 4008 and includes tether 4006 and burr cap 4004 or a proximal portion with other electronic components that are disposed against the skull. The illumination source, encapsulant, tether, burr cap and/or proximal portion may take the form of any of the examples described herein.

As discussed above, the devices, methods and systems may be used to treat tumors in the brain, Alzheimer's disease, Parkinson's disease, or any other neurodegenerative disease. However, this is not intended to be limiting and any diseased or damaged tissue may be treated using the device, methods, and systems disclosed herein.

Additionally, one of skill in the art will appreciate that any feature or element in any example may be used in combination with or substituted with another feature of any other example disclosed herein. Therefore, any permutation or combination of features or elements may be utilized.

### NOTES AND EXAMPLES

The following, non-limiting examples, detail certain aspects of the present subject matter to solve the challenges and provide the benefits discussed herein, among others.

Example 1 is a device for phototherapy, comprising: a light source configured to emit light; and an encapsulant encapsulating the light source, wherein the encapsulant is configured to isolate the light source from adjacent tissue.

Example 2 is the device of Example 1, wherein the encapsulant comprises a thin-film inorganic coating of aluminum oxide, Hafnium(IV) oxide, silicon dioxide, silicon carbide, diamond, polyimide, parylene, liquid crystal polymer, silicone elastomer, SU-8, cyclic olefin copolymer, crystalline ceramics, glass, sapphire, a polymer or a metal.

Example 3 is the device of any of Examples 1-2, wherein the encapsulant is configured to dissipate heat and control a rise in temperature of the device.

Example 4 is the device of any of Examples 1-3, further comprising an embedded material disposed in the encapsulant, the embedded material configured to disperse heat or act as an insulation material.

Example 5 is the device of any of Examples 1-4, wherein the encapsulant comprises optical properties that facilitate diffusion of the light emitted from the light source.

Example 6 is the device of any of Examples 1-5, wherein the light source comprises a light emitting diode (LED), a laser diode, an organic LED, or an optoelectronic device.

Example 7 is the device of any of Examples 1-6, wherein the light source emits light at a near infrared wavelength.

Example 8 is the device of any of Examples 1-7, wherein the light source is configured to emit light at a wavelength suitable for exciting a chromophore in a patient being treated for cancer or a neurodegenerative disease.

Example 9 is the device of any of Examples 1-8, further comprising one or more optical elements coupled to the encapsulant that have optical properties to modify the light emitted from the light source.

Example 10 is the device of any of Examples 1-9, wherein the optical elements comprise lenses or refractors.

Example 11 is the device of any of Examples 1-10, wherein the light source comprises a plurality of light sources emitting light in a different direction from one another.

Example 12 is the device of any of Examples 1-11, further comprising a lightguide structure optically coupled to the light source, and wherein the lightguide structure is configured to direct light to a desired direction, focus the light, or diffuse the light.

Example 13 is the device of any of Examples 1-12, further comprising a sensor configured to sense a condition adjacent the sensor, wherein the sensor is encapsulated by the encapsulant.

Example 14 is the device of any of Examples 1-13, wherein the sensor is a capacitive sensor.

Example 15 is the device of any of Examples 1-14, further comprising a tether coupled to the light source, and wherein the tether comprises a lumen configured for drug delivery.

Example 16 is the device of any of Examples 1-15, further comprising a radially expandable member having an expanded configuration and a collapsed configuration, wherein in the expanded configuration the radially expandable member is configured to support tissue adjacent thereto, and wherein the light source is disposed in the radially expandable member.

Example 17 is a system for phototherapy, comprising: the device of any of Examples 1-16; and a surgical delivery instrument.

Example 18 is the system of Example 17, further comprising a burr cap or a securement plate.

Example 19 is a method for phototherapy, comprising: illuminating a target treatment tissue with light emitted from a device comprising a light source, wherein the light source is encapsulated in an encapsulant; and maintaining isolation between the light source and the target treatment tissue with the encapsulant.

Example 20 is the method of Example 19, further comprising causing heat dissipation and controlling temperature with the encapsulant.

Example 21 is the method of any of Examples 19-20, wherein illuminating the target treatment tissue with the light emitted from the light source comprises emitting a wavelength suitable for exciting a chromophore in a patient being treated for cancer or a neurodegenerative disease.

Example 22 is the method of any of Examples 19-21, wherein illuminating the target treatment tissue with the light emitted from the light source comprises emitting the light from a light emitting diode (LED), a laser diode, an organic LED, or an optoelectronic device.

Example 23 is the method of any of Examples 19-22, wherein the encapsulant comprises optical properties that facilitate diffusion of the light emitted from the light source, the method further comprising causing diffusion of the light emitted from the light source.

Example 24 is the method of any of Examples 19-23, further comprising modifying the light emitted from the light source with optical elements coupled to the encapsulant, the optical elements comprising lenses, refractors, or a lightguide, that focus the light, diffuse the light or direct the light to a desired direction.

Example 25 is the method of any of Examples 19-24, wherein the device further comprises a sensor disposed in the encapsulant, the method further comprising sensing a condition adjacent the sensor.

Example 26 is the method of any of Examples 19-25, wherein the device further comprises a tether coupled to the light source, and wherein the tether comprises a lumen, the method further comprising delivering a drug to a target treatment tissue via the lumen.

Example 27 is the method of any of Examples 19-26, wherein the device further comprises a radially expandable member in which the light source is disposed, the method further comprising radially expanding the radially expandable member from a collapsed configuration to an expanded configuration, wherein in the expanded configuration the radially expandable member supports the target treatment tissue.

Example 28 is the method of any of Examples 19-27, wherein illuminating the target treatment tissue with the light comprises illuminating tissue in or adjacent the substantia nigra pars compacta tissue in the brain.

Example 29 is the method of any of Examples 19-28, wherein illuminating the target treatment tissue with the light comprises illuminating the target treatment tissue intraoperatively or after a surgical wound has been closed.

In Example 30, the devices, systems or methods of any one or any combination of Examples 1-29 can optionally be configured such that all elements or options recited are available to use or select from.

The above detailed description includes references to the accompanying drawings, which form a part of the detailed description. The drawings show, by way of illustration, specific embodiments in which the invention can be practiced.

These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A device for phototherapy, comprising:
a light source configured to emit light; and
an encapsulant encapsulating the light source, wherein the encapsulant is configured to isolate the light source from adjacent tissue.

2. The device of claim 1, wherein the encapsulant comprises a thin-film inorganic coating of aluminum oxide, Hafnium(IV) oxide, silicon dioxide, silicon carbide, diamond, polyimide, parylene, liquid crystal polymer, silicone elastomer, SU-8, cyclic olefin copolymer, crystalline ceramics, glass, sapphire, a polymer or a metal.

3. The device of claim 1, wherein the encapsulant is configured to dissipate heat and control a rise in temperature of the device.

4. The device of claim 1, further comprising an embedded material disposed in the encapsulant, the embedded material configured to disperse heat or act as an insulation material.

5. The device of claim 1, wherein the encapsulant comprises optical properties that facilitate diffusion of the light emitted from the light source.

6. The device of claim 1, wherein the light source comprises a light emitting diode (LED), a laser diode, an organic LED, or an optoelectronic device.

7. The device of claim 1, wherein the light source emits light at a near infrared wavelength.

8. The device of claim 1, wherein the light source is configured to emit light at a wavelength configured to emit light at a wavelength suitable for exciting a chromophore in a patient being treated for cancer or a neurodegenerative disease.

9. The device of claim 1, further comprising one or more optical elements coupled to the encapsulant that have optical properties to modify the light emitted from the light source.

10. The device of claim 9, wherein the optical elements comprise lenses or refractors.

11. The device of claim 1, wherein the light source comprises a plurality of light sources, each light source emitting light in a different direction from one another.

12. The device of claim 1, further comprising a lightguide structure optically coupled to the light source, and wherein the lightguide structure is configured to direct light to a desired direction, focus the light, or diffuse the light.

13. The device of claim 1, further comprising a sensor configured to sense a condition adjacent the sensor, wherein the sensor is encapsulated by the encapsulant.

14. The device of claim 13, wherein the sensor is a capacitive sensor.

15. The device of claim 1, further comprising a tether coupled to the light source, and wherein the tether comprises a lumen configured for drug delivery.

16. The device of claim 1, further comprising a radially expandable member having an expanded configuration and a collapsed configuration, wherein in the expanded configuration the radially expandable member is configured to support tissue adjacent thereto, and wherein the light source is disposed in the radially expandable member.

17. A system for phototherapy, comprising:
the device of claim 1; and
a surgical delivery instrument.

18. The system of claim 17, further comprising a burr cap or a securement plate.

19. A method for phototherapy, comprising:
illuminating a target treatment tissue with light emitted from a device comprising a light source, wherein the light source is encapsulated in an encapsulant; and
maintaining isolation between the light source and the target treatment tissue with the encapsulant.

20. The method of claim 19, further comprising causing heat dissipation and controlling temperature with the encapsulant.

21. The method of claim 19, wherein illuminating the target treatment tissue with the light emitted from the light source comprises emitting a wavelength suitable for exciting a chromophore in a patient being treated for cancer or a neurodegenerative disease.

22. The method of claim 19, wherein illuminating the target treatment tissue with the light emitted from the light source comprises emitting the light from a light emitting diode (LED), a laser diode, an organic LED, or an optoelectronic device.

23. The method of claim 19, wherein the encapsulant comprises optical properties that facilitate diffusion of the light emitted from the light source, the method further comprising causing diffusion of the light emitted from the light source.

24. The method of claim 19, further comprising modifying the light emitted from the light source with optical elements coupled to the encapsulant, the optical elements comprising lenses, refractors, or a lightguide, that focus the light, diffuse the light or direct the light to a desired direction.

25. The method of claim 19, wherein the device further comprises a sensor disposed in the encapsulant, the method further comprising sensing a condition adjacent the sensor.

26. The method of claim 19, wherein the device further comprises a tether coupled to the light source, and wherein the tether comprises a lumen, the method further comprising delivering a drug to a target treatment tissue via the lumen.

27. The method of claim 19, wherein the device further comprises a radially expandable member in which the light source is disposed, the method further comprising radially expanding the radially expandable member from a collapsed configuration to an expanded configuration, wherein in the expanded configuration the radially expandable member supports the target treatment tissue.

28. The method of claim 19, wherein illuminating the target treatment tissue with the light comprises illuminating tissue in or adjacent the substantia nigra pars compacta tissue in the brain.

29. The method of claim 19, wherein illuminating the target treatment tissue with the light comprises illuminating the target treatment tissue intraoperatively or after a surgical wound has been closed.
